(19) United States
(12) Patent Application Publication
(10) Pub. No.: US 2024/0150740 A1
(43) Pub. Date: May 9, 2024

(54) MOBILE ENDONUCLEASES FOR HERITABLE MUTATIONS

(71) Applicant: Keygene N.V., Wageningen (NL)

(72) Inventors: Julia Johanna WIND, Wageningen (NL); Bjorn Alexander KLOOSTERMAN, Wageningen (NL); Franck Georges Paul LHUISSIER, Wageningen (NL)

(73) Assignee: Keygene N.V., Wageningen (NL)

(21) Appl. No.: 18/487,025

(22) Filed: Oct. 13, 2023

Related U.S. Application Data
(63) Continuation of application No. PCT/EP2022/060160, filed on Apr. 15, 2022.

(30) Foreign Application Priority Data
Apr. 15, 2021 (EP) 21168638.1

(51) Int. Cl. C12N 9/22; C12N 15/74; C12N 15/82; C12N 15/86

(52) U.S. Cl. CPC C12N 9/22; C12N 15/743; C12N 15/8229; C12N 15/86; C12N 2310/20; C12N 2770/00043

(57) ABSTRACT

The invention concerns the targeted genomic modification of a plant cell, preferably a meristem cell. More in particular, the invention pertains to a vector expressing a coding RNA, wherein the coding RNA comprises a sequence encoding a CRISPR-nuclease and a mobile element, wherein the mobile element enables intercellular translocation of the coding RNA, preferably intercellular translocation to a meristem cell. The invention further concerns an editing RNA comprising the coding RNA and further comprising a guide RNA.

### CROSS-REFERENCE TO RELATED APPLICATIONS

This application is a continuation of International Patent Application No. PCT/EP2022/060160 filed Apr. 15, 2022, which application claims priority to European Patent Application No. 21168638.1 filed Apr. 15, 2021, the contents of which are all incorporated herein by reference in their entireties.

### SEQUENCE LISTING

The present application is filed along with a Sequence Listing in electronic format. The Sequence Listing is provided as a file entitled 085342-4700.xml, created on Jan. 28, 2024, which is 77,503 bytes in size. The information in the electronic format of the Sequence Listing is incorporated herein by reference in its entirety.

### FIELD OF THE INVENTION

The present invention relates to the field of molecular plant biology. The invention concerns the targeted genetic modification of a plant cell. In particular, the invention concerns the targeted modification of a plant meristem cell as well as plants derived therefrom.

### BACKGROUND

Making quick and efficient directed and heritable mutations in crops is important for the study of traits and gene validation. Introducing edits in intact meristems is considered highly desirable as it ensures propagation into the next generation. Creating edits in the meristem can be approached in two manners: by getting an editing complex (e.g. CAS9 with guide RNAs) in the meristem and edit these cells directly, or by editing somatic tissues and trigger meristem formation afterwards.

Edited somatic cells are converted to edited meristems, resulting in edits being transmitted to the progeny. However, this method would not work in those crops where meristem formation is hard or impossible to trigger through in vitro tissue culture, such as in bell pepper.

Unfortunately, the direct delivery of transgenes to meristem has been proven difficult, and so far has only been successful in Arabidopsis thaliana and its close relatives (Clough, S J and Bent, A F, “Floral dip: a simplified method for Agrobacterium-mediated transformation of Arabidopsis thaliana”. Plant J. 16, 735-743 (1998), and Hamada, H. et al. “An in planta biolistic method for stable wheat transformation. Sci. Rep. 7, 11443 (2017)).

There is therefore still a need in the art for a method to achieve efficient and heritable mutations in plants, without the need of regeneration.

Previously, it has been shown in the art that heritable mutations could be achieved by transfecting a Cas9 transgenic N. benthamiana plant with a vector expressing one or more mobile guide RNAs. Transfection of these guide RNAs resulted in heritable gene editing (Ellison et al, Multiplexed heritable gene editing using RNA viruses and mobile single guide RNAs, Nat Plants, 2020; 6(6):620-624).

Unfortunately, this approach necessitated the use of a Cas9 transgenic plant for the production of heritable mutations. It may require elaborate experimentation to produce such transgenic plant, or may not be feasible at all. Moreover, any seeds produced from such plants may comprise the heritable mutation but also still express the transgenic Cas9 protein. Amongst others, such transgenic plants may be difficult to commercialize for e.g. food consumption.

There thus remains a need in the art for a universal method to efficiently produce non-transgenic plants comprising heritable mutations. There is further a need for nucleic acids and vectors for use in such method.

### SUMMARY

The invention can be summarized in the following embodiments:

Embodiment 1. A vector expressing a coding RNA, wherein the coding RNA comprises a sequence encoding a CRISPR-nuclease and a first mobile element, wherein the mobile element enables intercellular translocation of the coding RNA and wherein preferably the CRISPR-nuclease comprises a nuclear localization signal.

Embodiment 2. A vector according to embodiment 1, wherein the vector is
a) a virus, wherein preferably the virus does not express a functional coat protein, preferably the virus has a deletion in a sequence encoding the coat protein;
b) a naked DNA, wherein the naked DNA is preferably a circular nucleic acid molecule; or
c) a DNA complex, wherein preferably the DNA molecule is coupled to a carrier, wherein the carrier is preferably selected from the group consisting of a lipoplex, a liposome, a polymersome, a polyplex, PEG, a dendrimer, an inorganic nanoparticle, a virosome and cell-penetrating peptides.

Embodiment 3. A vector according to embodiment 2, wherein the vector is a Tobacco Rattle Virus (TRV), a Tobacco Mosaic Virus (TMV) or a Sonchus yellow net virus (SYNV), preferably a tobacco mosaic virus RNA-based overexpression vector (TRBO).

Embodiment 4. A vector according to any one of the preceding embodiments, wherein the mobile element enables intercellular translocation to a meristem cell, preferably a shoot apical meristem cell.

Embodiment 5. A vector according to any one of the preceding embodiments, wherein the mobile element is at least one of a transfer-RNA (tRNA) and a gene transcript.

Embodiment 6. A vector according to embodiment 5, wherein at least one of:
the tRNA is at least one of a methionine, glycine-, threonine-, arginine-, lysine- and glutamine-tRNA; and
the gene transcript is a transcript of a gene selected from the group consisting of FT, GAI, SP2G, SP3D, SP5G, SP9D, CEN-like protein 1, protein MOTHER of FT and TF 1, Flowering locus T-a, Flowering locus T-b, PP16-1, GAIP, SCARECROW-LIKE (SCL14P), SHOOT MERISTEMLESS (STMP), ETHYLENE RESPONSE FACTOR (ERFP) and Myb (MybP), wherein optionally the FT gene transcript is a mutant FT and/or truncated FT.

Embodiment 7. A vector according to any one of the preceding embodiments, wherein the vector further comprises a guide RNA and optionally a second mobile element enabling intercellular translocation of the guide RNA.

Embodiment 8. An editing RNA comprising the coding RNA as defined in any one of embodiments 1 and 4-6, and further comprising the guide RNA, and optionally the second mobile element, as defined in embodiment 7.

Embodiment 9. An editing RNA according to embodiment 8, further comprising a cleavable spacer sequence located in between the coding RNA and the guide RNA.

Embodiment 10. An editing RNA according to embodiment 8 or 9, wherein at least one of the first and second mobile element is located at the 5′-end or at the 3′-end of the editing RNA.

Embodiment 11. An editing RNA according to any one of embodiments 8-10, wherein the editing RNA comprises two or more guide RNAs.

Embodiment 12. An editing RNA according to embodiment 11, wherein a cleavable spacer sequence is located in between the two or more guide RNAs.

Embodiment 13. An editing RNA according to embodiment 11 or 12, wherein the two or more guide RNAs direct the CRISPR-nuclease to the same gene.

Embodiment 14. A vector expressing an editing RNA as defined in any one of embodiments 8-13.

Embodiment 15. An agrobacterium expressing at least one of
i) the vector according to any one of embodiments 1-7 and 14; and
ii) an editing RNA according to any one of embodiments 8-13.

Embodiment 16. A method for producing a meristem cell having a targeted genomic modification, wherein the method comprises the steps of:
i) providing a plant; and
ii) expressing in a cell of the plant a coding RNA as defined in any one of embodiments 1 and 4-6, a guide RNA and optionally a second mobile element, as defined in embodiment 7,
   wherein the coding RNA and guide RNA translocate to a meristem cell,
   wherein the coding RNA and the guide RNA are comprised within an editing RNA according to any one of embodiments 8-13 and/or wherein the guide RNA is linked to the second mobile element, and
   wherein in the meristem cell a CRISPR-nuclease is expressed from the coding RNA and wherein the guide RNA directs the expressed CRISPR-nuclease to a location in the genome to generate a targeted genomic modification in the meristem cell.

Embodiment 17. A method according to embodiment 16, wherein the coding RNA and guide RNA are expressed by transfecting the plant cell with at least one of
a vector according to any one of embodiments 1-7 and 14; and
an agrobacterium as defined in embodiment 15.

Embodiment 18. A method according to embodiment 16 or 17, wherein the meristem cell is a shoot apical meristem cell.

Embodiment 19. A meristem cell having a targeted genomic modification wherein the cell is obtainable by the method according to any one of embodiments 16-18.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein.

It is clear for the skilled person that any methods and materials similar or equivalent to those described herein can be used for practising the present invention.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N. Y., 1989; Ausubel et al. Current Protocols in Molecular Biology, John Wiley & Sons, New York, 1987 and periodic updates; and the series Methods in Enzymology, Academic Press, San Diego.

The singular terms “a,” “an,” and “the” include plural referents unless the context clearly dictates otherwise. Thus, for example, reference to “a cell” includes a combination of two or more cells, and the like. The indefinite article “a” or “an” thus usually means “at least one”.

The term “and/or” refers to a situation wherein one or more of the stated cases may occur, alone or in combination with at least one of the stated cases, up to with all of the stated cases.

As used herein, the term “about” is used to describe and account for small variations. For example, the term can refer to less than or equal to ±(+ or −) 10%, such as less than or equal to ±5%, less than or equal to ±4%, less than or equal to +3%, less than or equal to +2%, less than or equal to +1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “comprising” is construed as being inclusive and open ended, and not exclusive. Specifically, the term and variations thereof mean the specified features, steps or components are included. These terms are not to be interpreted to exclude the presence of other features, steps or components.

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein”. An “isolated protein” is used to refer to a protein which is no longer in its natural environment, for example in vitro or in a recombinant bacterial or plant host cell.

“Plant” refers to either the whole plant or to parts of a plant, such as tissue or organs (e.g. pollen, seeds, gametes, roots, leaves, flowers, flower buds, anthers, fruit, etc.) obtainable from the plant, as well as derivatives of any of these and progeny derived from such a plant by selfing or crossing. Non-limiting examples of plants include crop plants and cultivated plants, such as African eggplant, alliums, artichoke, asparagus, barley, beet, bell pepper, bitter gourd, bladder cherry, bottle gourd, cabbage, canola, carrot, cassava, cauliflower, celery, chicory, common bean, corn salad, cotton, cucumber, eggplant, endive, fennel, gherkin, grape, hot pepper, lettuce, maize, melon, oilseed rape, okra, parsley, parsnip, pepino, pepper, potato, pumpkin, radish, rice, ridge gourd, rocket, rye, snake gourd, sorghum, spinach, sponge gourd, squash, sugar beet, sugar cane, sunflower, tomatillo, tomato, tomato rootstock, vegetable Brassica, watermelon, wax gourd, wheat and zucchini.

“Plant cell(s)” include protoplasts, gametes, suspension cultures, microspores, pollen grains, etc., either in isolation or within a tissue, organ or organism. The plant cell can e.g. be part of a multicellular structure, such as a callus, meristem, plant organ or an explant.

“Similar conditions” for culturing the plant/plant cells means among other things the use of a similar temperature, humidity, nutrition and light conditions, and similar irrigation and day/night rhythm.

The terms “homology”, “sequence identity” and the like are used interchangeably herein. Sequence identity is herein defined as a relationship between two or more amino acid (polypeptide or protein) sequences or two or more nucleotide (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The percentage sequence identity/similarity can be determined over the full length of the sequence.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. “Percent identity” is the identity fraction times 100.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined herein). The percent of sequence identity is preferably determined using the “BESTFIT” or “GAP” program of the Sequence Analysis Software Package™ (Version 10; Genetics Computer Group, Inc., Madison, Wis.). GAP uses the Needleman and Wunsch global alignment algorithm (Needleman and Wunsch, Journal of Molecular Biology 48:443-453, 1970) to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). gap extension penalty is 0.5; default scoring matrices are Blossum62 for proteins and DNAFull for DNA). “BESTFIT” performs an optimal alignment of the best segment of similarity between two sequences and inserts gaps to maximize the number of matches using the local homology algorithm of Smith and Waterman (Smith and Waterman, Advances in Applied Mathematics, 2:482-489, 1981, Smith et al., Nucleic Acids Research 11:2205-2220, 1983). When sequences have a substantially different overall lengths, local alignments, such as those using the Smith Waterman algorithm, are preferred.

Useful methods for determining sequence identity are also disclosed in Guide to Huge Computers, Martin J. Bishop, ed., Academic Press, San Diego, 1994, and Carillo, H., and Lipton, D., Applied Math (1988) 48:1073. More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

“Analogous to” in respect of a domain, sequence or position of a protein, in relation to an indicated domain, sequence or position of a reference protein, is to be understood herein as a domain, sequence or position that aligns to the indicated domain, sequence or position of the reference protein, upon alignment of the protein to the reference protein using alignment algorithms as described herein, such as Needleman Wunsch.

“Analogous to” in respect of a domain, sequence or position of a nucleic acid, in relation to an indicated domain, sequence or position of a reference nucleic acid, is to be understood herein as a domain, sequence or position that aligns to the indicated domain, sequence or position of the reference nucleic acid, upon alignment of the nucleic acid to the reference nucleic acid using alignment algorithms as described herein, such as Needleman Wunsch.

A “nucleic acid” or “polynucleotide” according to the present invention may include any polymer or oligomer of pyrimidine and purine bases, preferably cytosine, thymine, and uracil, and adenine and guanine, respectively (See Albert L. Lehninger, Principles of Biochemistry, at 793-800 (Worth Pub. 1982) which is herein incorporated by reference in its entirety for all purposes). The present invention contemplates any deoxyribonucleotide, ribonucleotide or nucleic acid component, and any chemical variants thereof, such as methylated, hydroxymethylated or glycosylated forms of these bases, and the like. The polymers or oligomers may be heterogeneous or homogenous in composition, and may be isolated from naturally occurring sources or may be artificially or synthetically produced. In addition, the nucleic acids may be DNA (optionally cDNA) or RNA, or a mixture thereof, and may exist permanently or transitionally in single-stranded or double-stranded form, including homoduplex, heteroduplex, and hybrid states. An “isolated nucleic acid” is used to refer to a nucleic acid which is no longer in its natural environment, for example in vitro or in a recombinant bacterial or plant cell.

The nucleic acid and/or protein of the invention may be at least one of a recombinant, synthetic or artificial nucleic acid and/or protein.

The terms “nucleic acid construct”, “nucleic acid vector”, “vector” and “expression construct” are used interchangeably herein and is herein defined as a man-made nucleic acid molecule resulting from the use of recombinant DNA technology. The terms “nucleic acid construct” and “nucleic acid vector” therefore does not include naturally occurring nucleic acid molecules although a nucleic acid construct may comprise (parts of) naturally occurring nucleic acid molecules.

The vector backbone may for example be a binary or superbinary vector (see e.g. U.S. Pat. No. 5,591,616, US 2002138879 and WO 95/06722), a co-integrate vector or a T-DNA vector, as known in the art and as described elsewhere herein, into which a chimeric gene is integrated or, if a suitable transcription regulatory sequence is already present, only a desired nucleic acid sequence (e.g. a coding sequence, an antisense or an inverted repeat sequence) is integrated downstream of the transcription regulatory sequence. Vectors can comprise further genetic elements to facilitate their use in molecular cloning, such as e.g. selectable markers, multiple cloning sites and the like.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′ non-translated sequence (3′ end) comprising a polyadenylation site.

“Expression of a gene” refers to the process wherein a DNA region which is operably linked to appropriate regulatory regions, particularly a promoter, is transcribed into an RNA, which is biologically active, e.g. which is capable of being translated into a biologically active protein or peptide, or e.g. a regulatory non-coding RNA.

The term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked may mean that the DNA sequences being linked are contiguous.

“Promoter” refers to a nucleic acid fragment that functions to control the transcription of one or more nucleic acids. A promoter fragment is preferably located upstream (5′) with respect to the direction of transcription of the transcription initiation site of the gene, and is structurally identified by the presence of a binding site for DNA-dependent RNA polymerase, transcription initiation site(s) and can further comprise any other DNA sequences, including, but not limited to transcription factor binding sites, repressor and activator protein binding sites, and any other sequences of nucleotides known to one of skill in the art to act directly or indirectly to regulate the amount of transcription from the promoter.

A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells.

Optionally the term “promoter” may also include the 5′ UTR region (5′ Untranslated Region) (e.g. the promoter may herein include one or more parts upstream of the translation initiation codon of transcribed region, as this region may have a role in regulating transcription and/or translation).

A “3′ UTR” or “3′ non-translated sequence” (also often referred to as 3′ untranslated region, or 3′end) refers to the nucleic acid sequence found downstream of the coding sequence of a gene, which comprises for example a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

The term “cDNA” means complementary DNA. Complementary DNA is made by reverse transcribing RNA into a complementary DNA sequence. cDNA sequences thus correspond to RNA sequences that are expressed from genes. As RNA sequences expressed from the genome can undergo splicing, i.e. introns are spliced out of the pre-mRNA and exons are joined together, before being translated in the cytoplasm into proteins, it is understood that the sequence of the cDNA corresponds to the sequence of the mRNA. The cDNA sequence thus may not be identical to the genomic DNA sequence to which it corresponds as the cDNA may encode only the complete open reading frame, consisting of the joined exons, for a protein, whereas the genomic DNA sequence may comprise exon sequences interspersed by intron sequences. Genetically modifying a gene which encodes a protein may thus not only relate to modifying the sequences encoding the protein, but may also involve mutating intronic sequences of the genomic DNA and/or other gene regulatory sequences of that gene.

The term “regeneration” is herein defined as the formation of a new tissue and/or a new organ from a single plant cell, a callus, an explant, a tissue or from an organ. The regeneration pathway can be somatic embryogenesis or organogenesis. Somatic embryogenesis is understood herein as the formation of somatic embryos, which can be grown to regenerate whole plants. Organogenesis is understood herein as the formation of new organs from (undifferentiated) cells. Preferably, the regeneration is at least one of ectopic apical meristem formation, shoot regeneration and root regeneration.

The regeneration as defined herein can preferably concern at least de novo shoot formation. For example, regeneration can be the regeneration of a(n) (elongated) hypocotyl explant towards a(n) (inflorescence) shoot.

Regeneration may further include the formation of a new plant from a single plant cell or from e.g. a callus, an explant, a tissue or an organ. The regeneration process can occur directly from parental tissues or indirectly, e.g. via the formation of a callus.

The term “conditions that allow for regeneration” is herein understood as an environment wherein a plant cell or tissue can regenerate. Such conditions include at minimum a suitable temperature (i.e. between 0° C.-60° C.), nutrition, day/night rhythm, irrigation and one or more plant hormones and/or plant hormone-like compounds. Furthermore, “optimal conditions that allow for regeneration” are those environmental conditions that allow for a maximum regeneration of the plant cells.

The term “wild type” as used in the context of the present invention in combination with a protein or nucleic acid means that said protein or nucleic acid consists of an amino acid or nucleotide sequence, respectively, that occurs as a whole in nature and can be isolated from organisms in nature as such, e.g. is not the result of modification techniques such as targeted or random mutagenesis or the like. A wild type protein is expressed in at least a particular developmental stage under particular environmental conditions, e.g. as it occurs in nature.

The term “endogenous” as used in the context of the present invention in combination with a protein or nucleic acid means that said protein or nucleic acid is still contained within the plant, i.e. is present in its natural environment. Often an endogenous gene will be present in its normal genetic context in the plant.

Targeted mutagenesis is mutagenesis that can be designed to alter a specific nucleotides or nucleic acid sequence, such as but not limited to, oligo-directed mutagenesis, RNA-guided endonucleases (e.g. the CRISPR-technology), TALENs or Zinc finger technology.

The term “sequence of interest” includes, but is not limited to, any genetic sequence preferably present within a cell, such as, for example a gene, part of a gene, or a non-coding sequence within or adjacent to a gene. The sequence of interest may be present in a chromosome, an episome, an organellar genome such as mitochondrial or chloroplast genome or genetic material that can exist independently to the main body of genetic material such as an infecting viral genome, plasmids, episomes, transposons for example. A sequence of interest may be within the coding sequence of a gene, within transcribed non-coding sequence such as, for example, leader sequences, trailer sequence or introns. Said sequence of interest may be present in a double or a single strand nucleic acid molecule. The nucleic acid sequence is preferably present in a double-stranded nucleic acid molecule. The sequence of interest may be any sequence within a nucleic acid, e.g., a gene, gene complex, locus, pseudogene, regulatory region, highly repetitive region, polymorphic region, or portion thereof. The sequence of interest may also be a region comprising genetic or epigenetic variations indicative for a phenotype or disease. Preferably, the sequence of interest is a small or longer contiguous stretch of nucleotides (i.e. a polynucleotide) of duplex DNA, wherein said duplex DNA further comprises a sequence complementary to the target sequence in the complementary strand of said duplex DNA.

A “control plant” as referred to herein is a plant of the same species and preferably same genetic background as the plant of the invention, i.e. a plant that has been subjected to the methods as taught herein. Preferably, the control plant only differs from the putative test plant in that the control plant does not have the targeted modification as detailed herein.

Preferably the control plant is grown under the same conditions as the plant subjected to a method of the invention.

### DETAILED DESCRIPTION

The inventors have discovered an approach in which the CRISPR-protein editing complex can enter an existing meristem, and as such bypasses the need for regeneration. To this end, the inventors have fused mobile elements to guide RNAs as well as to (m)RNAs encoding a CRISPR-protein, thereby creating RNA molecules that can travel through the plant transport system and enter the meristem.

The mobile RNAs can thus be introduced at any random suitable location in the plant tissue. These RNA molecules are subsequently loaded into the vascular tissues, and transported towards e.g. the meristem. Alternatively or in addition, these RNA molecules are transported through the plasmodesmata towards e.g. the meristem. Upon arrival in the target cells, the CRISPR-protein encoding RNA is translated into a CRISPR-protein, and combined with the guide RNA to form the necessary editing complex. When the edits are made in the meristem tissue, these edits will be carried to the subsequent generation(s).

In a first aspect, the invention therefore pertains to a coding RNA comprising or consisting of a sequence encoding a CRISPR-protein and a mobile element. The coding RNA may be synthetic or recombinant. The coding RNA may be a double- or single-stranded ribonucleic acid molecule consisting of or comprising a sequence encoding a CRISPR-protein and a mobile element. Optionally, the coding RNA is comprised in a vector, a construct or an editing RNA as defined herein. Preferably, the mobile element enables intercellular translocation of the coding RNA. Preferably, the CRISPR-protein comprises a nuclear localization signal. Preferably, the CRISPR-protein is a CRISPR-nuclease.

The intercellular translocation is understood herein as the long distance transport of a molecule, such as an RNA molecule as defined herein, from one cell to another cell. A preferred cell-to-cell movement is through phloem, xylem and/or plasmodesmata, preferably through phloem.

Water and mineral nutrients are transported from roots to the aerial parts of plants through the xylem. Phloem supports the movement of photosynthates and macromolecules from e.g. source to sink tissues. The phloem is composed of living enucleated sieve elements assisted by companion cells. These sieve element cells stack together to form the sieve tube, which allows for rapid flow of molecules over long distances in plants. Macromolecules, including proteins, RNAs and ribonucleoprotein complexes, have been found in the phloem stream.

Interestingly, RNase activity is not detectable in phloem sap. Indeed, various RNA species are likely transported through the phloem to distant tissues (Liu et al, Nat Plants. 2018; 4(11): 869-878).

### A Coding RNA

The coding RNA may be introduced in a first plant cell and the mobile element directs the coding RNA into the vascular tissues of the plant. The coding RNA travels through the vascular tissues to another, second, plant cell, where the CRISPR-protein is translated from the coding RNA. Alternatively or in addition, the coding RNA uses cell-to-cell transport through plasmodesmata to travel to another, second, plant cell.

The term “vascular tissues” of a plant is well-known to the skilled person and encompasses both xylem and phloem. A preferred vascular tissue is the phloem. Thus preferably the mobile element directs the RNA into the phloem of a plant.

Upon arrival of the coding RNA into a second or “target” cell, the CRISPR-protein is expressed and, upon forming a complex with the guide RNA, can modify a target sequence in the second cell.

The sequence of the coding RNA preferably at least comprises the sequence encoding the CRISPR-protein and the sequence of the mobile element. The coding RNA may comprise additional sequences. As a non-limiting example, the CRISPR-protein encoding sequence may be flanked by at least one of a 5′-UTR and a 3′-UTR. The coding RNA may comprise additional sequences, such as, but not limited to, one or more sequences that augment or aid in the translation of the CRISPR-protein from the coding RNA. The coding RNA may comprise sequences that aid in the initiation and/or aid in the termination of translation. The CRISPR-protein encoding sequence may comprise its wild type 3′-UTR and/or wild type 5′-UTR sequence. In addition or alternatively, the CRISPR-protein encoding sequence is followed by e.g. a T-repeat sequence.

Preferably, the sequence encoding the CRISR-protein is optimized for expression in plant cells, i.e. codon-optimized for expression in plant cells. In addition or alternatively, the sequence coding for the CRISPR-protein may comprise intronic sequences. Preferably, the sequence encoding the CRISPR-protein is devoid of intronic sequences.

The coding RNA preferably at least comprises a mobile element and a sequence encoding the CRISPR-protein. The coding RNA may comprise a single sequence encoding a CRISPR-protein and/or a single mobile element. Alternatively, the coding RNA may comprise multiple sequences encoding a CRISPR-protein and/or multiple mobile elements. The coding RNA may comprise 2, 3, 4, 5, 6, 7, 8, 9, 10 or more sequences encoding a CRISPR-protein and/or 2, 3, 4, 5, 6, 7, 8, 9, 10 or more mobile elements. Preferably, the coding RNA comprises at least two mobile elements flanking the sequence encoding the CRISPR-protein.

The coding RNA may comprise a coding sequence for two or more different type of proteins, such as, but not limited to, the coding sequence for 2, 3, 4, 5, 6 or more different proteins. As a non-limiting example, the coding RNA may comprise one or more CRISPR-protein encoding sequences and one or more reporter protein encoding sequences, such as a sequence encoding a Green Fluorescence Protein (GFP).

### Mobile Element

The mobile element as used in the invention is an element that is preferably comprised in at least one of the coding RNA the editing RNA and the guiding RNA as defined herein.

An editing RNA is to be understood herein as comprising two elements, wherein the first element is a coding RNA as defined herein, and the second element is a guide RNA. Optionally, the second element is a guiding RNA as defined herein. The editing RNA may be synthetic or recombinant. The editing RNA may be a double- or single-stranded ribonucleic acid molecule consisting of or comprising a coding RNA and a guide RNA. Optionally, the editing RNA is comprised in a vector or a construct.

A guiding RNA is to be understood herein as comprising or consisting of a mobile element and a guide RNA. The guiding RNA may be synthetic or recombinant. The guiding RNA may be a double- or single-stranded ribonucleic acid molecule consisting of or comprising a guide RNA and a mobile element. Optionally, the guiding RNA is comprised in a a vector, a construct or an editing RNA. Optionally, the editing RNA comprises a coding RNA and a guiding RNA.

Any mobile element that results in intercellular translocation of an RNA molecule will be suitable for use in the invention. The mobile element may translocate the RNA molecule from a first “production” cell into the vascular tissue of a plant and from the vascular tissue into the second “target” cell. The mobile element preferably translocates the RNA molecule into the xylem and/or phloem, preferably into the phloem of a plant.

Alternatively or in addition, the mobile element may use cell-to-cell transport through plasmodesmata to translocate the RNA molecule to the second “target” plant cell.

There are mobile RNA elements known in the art that can transport RNA molecules to specific plant tissues, such as the meristem tissue, root tissue and/or leaves. Preferably, the mobile element translocates the RNA molecule to the meristem tissue. However, the invention is not limited to mobile elements that translocate the RNA molecule to a meristem tissue. As a non-limiting example, the mobile elements may additionally or alternatively translocate the RNA molecules to the gametes, leaves and/or flowers of a plant. The cells comprising a modification may subsequently be isolated and regenerated into a new plant.

Preferably, the mobile element is capable of translocating the RNA molecule to a meristem cell. The mobile element for use in the invention thus preferably translocates the RNA molecule from a first “production” cell to a second “target” cell, wherein the second cell is preferably a meristem cell. Interestingly, it has been shown previously that mobile RNA elements are recognized by plant transport proteins, which ensures protection from degradation of the RNA that is attached to these mobile elements during transport (Ham et al, 2009, Plant Cell (2009); 21(1):197-215).

The meristem is a tissue comprising undifferentiated cells that are capable of cell division. These cells are totipotent and can develop into all other tissues and organs that occur in plants: the root meristem cells form root organs and the shoot meristem cells form shoot organs. The mobile element preferably translocates the RNA molecule to a meristem cell, wherein the meristem cell preferably is at least one of an apical meristem cell, an intercalary meristem cell and a floral meristem cell. Preferably, the mobile element translocates the RNA molecule to an apical meristem cell. The apical meristem cell is preferably a shoot apical meristem cell or a root apical meristem cell. Preferably, the mobile element translocates the RNA molecule to a shoot apical meristem cell (SAM). The shoot apical meristem is the source of all above-ground organs, such as, but not limited to, the leaves and flowers. The shoot apical meristem is also the site of most of the embryogenesis in plants. The meristem cell, preferably the shoot apical meristem cell, may develop into an egg cell or pollen. The mobile element for use in the invention preferably translocates the RNA molecule to a meristem cell, preferably a shoot apical meristem cell, wherein the shoot apical meristem cell preferably develops into an egg cell and/or pollen. It is understood herein that the phrase “the mobile element translocates” may be used interchangeably with the phrase “the mobile element enables the translocation”.

Preferably, the coding RNA of the invention comprises a mobile element, wherein the mobile element enables intercellular translocation to a meristem cell. Preferably, the coding RNA of the invention comprises a mobile element that enables translocation to a shoot apical meristem cell. The translocation of the RNA molecules of the invention into the second cell results in targeted genomic modifications in the second cell. In a non-limiting example, the target cell comprising the targeted genomic modification is a shoot apical meristem cell that develops into egg cells and/or pollen, The egg cells and/or pollen thus comprises the targeted genomic modification and it is carried on to the next generation.

The mobile element or elements for use in the invention may be located 5′ and/or 3′ of the sequence encoding the CRISPR-protein. In addition or alternatively, the mobile elements may be located 5′ and/or 3′ of the guide RNA.

The skilled person understands that any mobile element enabling intercellular translocation of the coding RNA, and optionally the guide RNA and/or editing RNA, would be suitable for use in the invention. The mobile element may be at least one of a transfer-RNA (tRNA) and a gene transcript, or a functional fragment thereof. Examples of such mobile elements are e.g. disclosed in Zhang W, et al (2016) tRNA-Related Sequences Trigger Systemic mRNA Transport in Plants, Plant Cell. doi: 10.1105/tpc. 15.01056 and Ellison E E et al, (2020) Multiplexed heritable gene editing using RNA viruses and mobile single guide RNAs. Nat Plants. doi: 10.1038/s41477-020-0670-y.

The tRNA may be a tRNA-like structure. Preferably, the tRNA, or tRNA-like structure is selected from the group consisting of tRNA^{Ala}, tRNA^{Arg}, tRNA^{Asn}, tRNA^{Asp}, tRNA^{Cys}, tRNA^{Gln}, tRNA^{Glu}, tRNA^{Gly}, tRNA^{His}, tRNA^{lle}, tRNA^{Leu}, tRNA^{Lys}, tRNA^{Met}, tRNA^{phe}, tRNA^{Pro}, tRNA^{Ser}, tRNA^{Thr}, tRNA^{Trp}, tRNA^{Tyr} and tRNA^{Val}. Alternatively, the tRNA-like structure can be selected from the group consisting of tRNA^{Ala}, tRNA^{Arg}, tRNA^{Asn}, tRNA^{Asp}, tRNA^{Cys}, tRNA^{Gln}, tRNA^{Glu}, tRNA^{Gly}, tRNA^{His}, tRNA^{lle}, tRNA^{Leu}, tRNA^{Lys}, tRNA^{Met}, tRNA^{phe}, tRNA^{Pro}, tRNA^{Ser}, tRNA^{Thr}, tRNA^{Trp}, tRNA^{Tyr} and tRNA^{Val} and wherein the tRNA-like structure lacks at least one of the D stem-loop, the D and T stem-loops and the D and A stem-loops. Preferably, the tRNA, or tRNA-like structure is selected from the group consisting of tRNA^{Arg}, tRNA^{Glu}, tRNA^{Gly}, tRNA^{Lys}, tRNA^{Met}, and tRNA^{Thr}. Alternatively, the tRNA-like structure can be selected from the group consisting of tRNA^{Arg}, tRNA^{Glu}, tRNA^{Gly}, tRNA^{Lys}, tRNA^{Met}, and tRNA^{Thr}, and wherein the tRNA-like structure lacks at least one of the D stem-loop, the D and T stem-loops and the D and A stem-loops.

The tRNA, or tRNA-like structure, may function as a cleavable mobile element. Hence in an embodiment, the tRNA, or tRNA-like structure as defined herein thus functions as a mobile element as defined herein as well as a cleavable sequence as defined herein.

The tRNA or tRNA-like structure for use in the invention may be a naturally occurring tRNA, having the ability to function as a mobile element as well as a cleavable sequence, i.e. to function as a cleavable mobile element. Any naturally occurring plant tRNA may have the ability to function as a cleavable sequence. Hence any tRNA or tRNA-like structure that functions as a mobile element, may additionally function as a cleavable sequence, e.g. functions as a cleavable mobile element. The skilled person understands how to straightforwardly obtain the sequence of such plant tRNA or tRNA-like structure.

The naturally occurring tRNA may comprise a recognition site for cleavage by at least one of RNase P and RNase Z. A naturally according tRNA may comprise the leader sequence AACAAA, preferably at, or in close vicinity of, the 3′- and/or 5′-end. The leader sequence may enable processing or “cleavage” of the mobile element. Preferably, the tRNA comprises the leader sequence AACAAA at, or in close vicinity of, the 5′-end of the tRNA. Suitable tRNA cleavable elements are e.g. described in Xie et al (“Boosting CRISPR/Cas9 multiplex editing capability with the endogenous tRNA-processing system”, 2015, Proc Natl Acad Sci USA; 112(11):3570-5), which is incorporated herein by reference. These cleavable tRNA sequences may thus additionally function as a mobile element as defined herein. A preferred cleavable tRNA is tRNA^{Gly}.

Alternatively or in addition, a tRNA or tRNA-like structure as defined herein can be modified to additionally function as a cleavable sequence. The tRNA or tRNA-like structure may be modified to comprise a recognition site for cleavage by at least one of RNase P and RNase Z. Preferably, the tRNA or tRNA-like structure is modified to comprise the leader sequence AACAAA, preferably at, or in close vicinity of, the 3′- and/or 5′-end. Preferably, the tRNA comprises the leader sequence AACAAA at, or in close vicinity of, the 5′-end of the tRNA.

A coding RNA, a guiding RNA and/or an editing RNA comprising a cleavable mobile element is preferably first translocated prior to cleavage in the second or “target” cell. The cleavable mobile element may be located within the coding RNA at, or in close vicinity of, the 3′- and/or 5′-end of said coding RNA. The cleavable mobile element may be located within the guiding RNA at, or in close vicinity of, the 3′-end and/or the 5′-end of said guiding RNA. Optionally, the cleavable mobile element may be located in between two or more guide RNAs comprised in a guiding RNA as defined herein. The cleavable mobile element may be located within the editing RNA at, or in close vicinity of, the 3′-end and/or the 5′-end of the editing RNA. As a non-limiting example, the cleavable mobile element may be located in between the guide RNA, optionally the guiding RNA, and the coding RNA that are comprised within the editing RNA. In addition or alternatively, the cleavable mobile element may be located in between one or more guide RNAs of the guiding RNA as defined herein.

The mobile element may be a gene transcript, or a functional fragment thereof. Preferably, the gene transcript is a transcript of a gene selected from the group consisting of FT, GAI, SP2G, SP3D, SP5G, SP9D, CEN-like protein 1, protein MOTHER of FT and TF 1, Flowering locus T-a and Flowering locus T-b, PP16-1, GAIP, SCARECROW-LIKE (SCL14P), SHOOT MERISTEMLESS (STMP), ETHYLENE RESPONSE FACTOR (ERFP) and Myb (MybP). The gene transcript may be a naturally occurring gene transcript, or a mutant, or a truncated gene transcript. Preferably, the mobile element is a gene transcript, wherein the gene transcript is at least one of FT, mutant FT and truncated FT. The gene transcript can be a naturally occurring gene transcript, e.g. the transcript is a transcript as listed in Table 1, or an homologue or orthologue thereof.

The gene transcript, or functional fragment thereof, may comprise at least one of a tRNA-like structure (TLS) motif and a Phosphotyrosine-binding (PTB) motif.

The gene transcript, or functional fragment thereof, may comprise a tRNA-like structure (TLS) motif. These structures are known in the art to trigger systemic transport of RNA molecules in plants (Zhang et al, Plant Cell. 2016; 28(6):1237-49. Examples of such mobile transcripts are listed in Zhang et al supra, supplemental dataset 1-3, which transcripts are incorporated herein by reference. The mobile element may be a dicistronic mRNA:tRNA gene transcript.

Alternatively or in addition, the gene transcript, or functional fragment thereof, may comprise a Phosphotyrosine-binding (PTB) motif. As a non-limiting example, genes from Cucurbita maxima cv Big Max: PP16-1, GAIP, SCARECROW-LIKE (SCL14P), SHOOT MERISTEMLESS (STMP), ETHYLENE RESPONSE FACTOR (ERFP), Myb (MybP) comprise a PTB binding motif and were shown in the art to be mobile and present in pumpkin phloem sap (Ham et al, supra)

TABLE 1
Examples of mobile elements

| Gene | | | SEQ |
|---|---|---|---|
| transcript¹ | Plant species | Gene ID | ID NO: |
| FT | Arabidopsis thaliana | AT1G65480.1 | 1 |
| GAI | Cucurbita maxima | LOC111472091 | 2 |
| SP2G | Solanum lycopersicum | Solyc02g079290.2.1 | 3 |
| SP3D | Solanum lycopersicum | Solyc03g063100.1.1 | 4 |
| SP5G | Solanum lycopersicum | Solyc05g053850.2.1 | 5 |
| SP9D | Solanum lycopersicum | Solyc11g008640.1.1 | 6 |
| CEN-like | Nicotiana sylvestris | LOC104248269 | 7 |
| protein 1 | | | |
| MOTHER of FT | Nicotiana sylvestris | LOC104210681 | 8 |
| and TF 1 | | | |
| Flowering locus | Nicotiana sylvestris | LOC104219896 | 9 |
| T-a | | | |
| Flowering locus | Nicotiana sylvestris | LOC104222479 | 10 |
| T-b | | | |
| PP16-1 | Cucurbita maxima cv | LOC111487707 | 11 |
| | Big Max | | |
| GAIP | Cucurbita maxima cv | LOC111472091 | 12 |
| | Big Max | | |
| SCARECROW- | Cucurbita maxima cv | LOC111499671 | 13 |
| LIKE (SCL14P) | Big Max | | |
| SHOOT MERI- | Cucurbita maxima cv | LOC111486097 | 14 |
| STEMLESS | Big Max | | |
| (STMP) | | | |
| ETHYLENE | Cucurbita maxima cv | | 15 |
| RESPONSE | Big Max | | |
| FACTOR (ERFP) | | | |
| Myb (MybP) | Cucurbita maxima cv | | 16 |
| | Big Max | | |
| stBel5 | Solanum tuberosum | | 28 |

¹See e.g. Li C et al (2009) A cis element within flowering locus T mRNA determines its mobility and facilitates trafficking of heterologous viral RNA. J Virol. doi: 10.1128/JVI.02346-08; Haywood V et al (2005) Phloem long-distance trafficking of GIBBERELLIC ACID-INSENSITIVE RNA regulates leaf development . Plant J. doi: 10.1111/j.1365-313X.2005.02351.x; Huang N C et al, (2018) Mobility of Antiflorigen and PEBP mRNAs in Tomato-Tobacco Heterografts. Plant Physiol. doi: 10.1104/pp.18.00725; and Banerjee, A. K. et al (2009) Untranslated regions of a mobile transcript mediate RNA metabolism. Plant Physiol. 151, 1831-1843

The mobile element for use in the invention may have at least 20%, 25%, 30%, 35%, 40%, 45%, 50%, 55%, 60%, 65%, 70%, 75%, 80%, 85%, 90%, 95%, 96%, 97%, 98%, 99% or 100% sequence identity with any one of SEQ ID NO: 1-16, and/or at least 20%, 25%, 30%, 35%, 40%, 45%, 50%, 55%, 60%, 65%, 70%, 75%, 80%, 85%, 90%, 95%, 96%, 97%, 98%, 99% or 100% sequence identity with any one of SEQ ID NO: 1-16 and 28. The mobile element may be a functional fragment of any one of SEQ ID NO: 1-16, and/or a functional fragment of any one of SEQ ID NO: 1-16 and 28.

### CRI SPR-Protein

The coding RNA preferably comprises a sequence encoding a CRISPR-protein, in addition to a mobile element. The sequence encoding the CRISPR-protein may be located 3′ or 5′ of the mobile element as defined herein. Preferably the codon sequence of the CRISPR-protein is optimized for expression in plant cells.

The CRISPR-protein is preferably at least one of a CRISPR-nuclease, CRISPR-nickase and a CRISPR-deaminase. Preferably, the CRISPR-protein is a CRISPR-nuclease.

The CRISPR-protein encoded by the coding RNA can be any suitable CRISPR-protein known in the art. Preferably, the CRISPR-protein encoded by the coding RNA comprises a nuclear localisation signal (NLS) to direct the expressed CRISPR-protein to the nucleus of the plant cell. The NLS may be located at the C-terminus and/or at the N-terminus of the CRISPR protein. Any known nuclear localisation signal would be suitable for use in the invention. Preferred nuclear localisation signals include, but are not limited to the NLS of the SV40 Large T-antigen MEDPTMAPKKKRKV (SEQ ID NO: 17), the monopartite NLS PKKKRKV (SEQ ID NO: 18) and the NLS of nucleoplasmin KRPAATKKAGQAKKKK (SEQ ID NO: 19). The CRISPR protein may comprise two or more nuclear localisation signals, e.g. one or more at the N-terminus and one or more at the C-terminus. The CRISPR-protein may comprise two or more different nuclear localisation signals, e.g. the NLS at the C-terminus may differ from the NLS at the N-terminus.

A CRISPR-nuclease comprises a nuclease domain and at least one domain that interacts with a guide RNA. When complexed with a guide RNA, the CRISPR-protein complex is directed to a specific nucleic acid sequence by a guide RNA. The guide RNA interacts with the CRISPR-nuclease as well as with a target-specific nucleic acid sequence, such that, once directed to the site comprising the target nucleic acid sequence via the guide sequence, the CRISPR-nuclease is able to introduce a double-stranded break at the target site.

In case the CRISPR-protein is a CRISPR-nuclease, both domains of the nuclease are catalytically active and the protein is able to introduce a double-stranded break at the target site.

In case the CRISPR-protein is a CRISPR-nickase, one domain of the nuclease is catalytically active and one domain is catalytically inactive, and the protein is able to introduce a single-stranded break at the target site.

The skilled person is well aware of how to design a guide RNA in a manner that it, when combined with a CRISPR-nuclease or CRISPR-nickase, effects the introduction of a single- or double-stranded break at a predefined site in the nucleic acid molecule.

CRISPR-proteins can generally be categorized into six major types (Type I-VI), which are further subdivided into subtypes, based on core element content and sequences (Makarova et al, 2011, Nat Rev Microbiol 9:467-77 and Wright et al, 2016, Cell 164(1-2):29-44). In general, the two key elements of a CRISPR-protein complex is a CRISPR-protein and a guide RNA.

Type II CRISPR-protein complexes include a signature Cas9 protein, a single protein (about 160 KDa), capable of generating crRNA and specifically cleaving duplex DNA. The Cas9 protein typically contains two nuclease domains, a RuvC-like nuclease domain near the amino terminus and the HNH (or McrA-like) nuclease domain near the middle of the protein. Each nuclease domain of the Cas9 protein is specialized for cutting one strand of the double helix (Jinek et al, 2012, Science 337 (6096): 816-821). The Cas9 protein is an example of a CRISPR-protein of the type II CRISPR/-CAS system and forms an endonuclease, when combined with the crRNA and a second RNA termed the trans-activating crRNA (tracrRNA). The crRNA and tracrRNA function together as the guide RNA. The CRISPR-nuclease complex introduces DNA double strand breaks (DSBs) at the position in the genome defined by the crRNA. Jinek et al. (2012, Science 337: 816-820) demonstrated that a single chain chimeric guide RNA (herein defined as a “sgRNA” or “single guide RNA”) produced by fusing an essential portion of the crRNA and tracrRNA was able to form a functional CRISPR-nuclease complex in combination with the Cas9 protein.

A Type V CRISPR-protein complex has been described, the Clustered Regularly Interspaced Short Palindromic Repeats from Prevotella and Francisella 1 or CRISPR/Cpf1. Cpf1 genes are associated with the CRISPR locus and coding for an endonuclease that use a crRNA to target DNA. Cpf1 is a smaller endonuclease than Cas9, which may overcome some of the CRISPR-Cas9 system limitations. Cpf1 is a single RNA-guided endonuclease lacking tracrRNA, and it utilizes a T-rich protospacer-adjacent motif. Cpf1 cleaves DNA via a staggered DNA double-stranded break (Zetsche et al (2015) Cell 163 (3): 759-771). The type V CRISPR-Protein system preferably includes at least one of Cpf1, C2c1 and C2c3.

The CRISPR-protein for use in the invention may comprise any CRISPR-protein as defined herein above. Preferably, the CRISPR-protein is a Type II CRISPR-protein, preferably a Type II CRISPR-nuclease, e.g., Cas9 (e.g., the protein of SEQ ID NO: 20], encoded by SEQ ID NO: 21, or the protein of SEQ ID NO: 22) or a Type V CRISPR-protein, preferably a Type V CRISPR-nuclease, e.g. Cpf1 (e.g., the protein of SEQ ID NO: 23, encoded by SEQ ID NO: 24) or Mad7 (e.g. the protein of SEQ ID NO: 25 or 26), or a protein derived thereof, having preferably at least about 70%, 80%, 90%, 91%, 92%, 93%, 94%, 95%, 96%, 97%, 98%, or 99% sequence identity to said protein over its whole length.

Preferably, the CRISPR-protein is a Type II CRISPR-nuclease, preferably a Cas9 nuclease.

The skilled person knows how to find and prepare a coding RNA comprising a sequence encoding the CRISPR-protein, such as a sequence encoding a CRISPR-nuclease. In the prior art, numerous reports are available on its design and use. See for example the review by Haeussler et al (J Genet Genomics. (2016)43(5):239-50. doi: 10.1016/j.jgg.2016.04.008.) on the design of guide RNA and its combined use with a CAS-protein (originally obtained from S. pyogenes), or the review by Lee et al. (Plant Biotechnology Journal (2016) 14(2) 448-462).

In general, a CRISPR-nuclease, such as Cas9, comprises two catalytically active nuclease domains. For example, a Cas9 protein can comprise a RuvC-like nuclease domain and an HNH-like nuclease domain. The RuvC and HNH domains work together, both cutting a single strand, to make a double-stranded break in DNA. (Jinek et al., Science, 337: 816-821).

A dead CRISPR-nuclease comprises modifications such that none of the nuclease domains shows cleavage activity. The CRISPR-nickase may be a variant of the CRISPR-nuclease wherein one of the nuclease domains is mutated such that it is no longer functional (i.e., the nuclease activity is absent). An example is a SpCas9 variant having either the D10A or H840A mutation. Preferably, the nuclease encoded by the coding RNA is not a dead nuclease.

Preferably, the CRISPR-protein is either a nickase or (endo)nuclease.

The CRISPR-protein encoded by the coding RNA may comprise or consist of a whole type II or type V CRISPR-protein or variant or functional fragment thereof. Preferably such fragment does bind the guide RNA, but e.g. may lack one or more residues required for nuclease activity.

Preferably, the CRISPR-protein encoded by the coding RNA of the invention is a Cas9 protein. The Cas9 protein may be derived from the bacteria Streptococcus pyogenes (SpCas9; NCBI Reference Sequence NC_017053.1; UniProtKB—Q99ZW2), Geobacillus thermodenitrificans (UniProtKB—A0A178TEJ9), Corynebacterium ulcerous (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria meningitidis (NCBI Ref: YP_002342100.1). Encompassed are Cas9 variants from these, having an inactivated HNH or RuvC domain homologues to SpCas9, e.g. the SpCas9_D10A or SpCas9_H840A, or a Cas9 having equivalent substitutions at positions corresponding to D10 or H840 in the SpCas9 protein, rendering a nickase.

The CRISPR-protein encoded by the coding RNA may be, or may be derived from, Cpf1, e.g. Cpf1 from Acidaminococcus sp; UniProtKB—U2UMQ6. The variant may be a Cpf1-nickase having an inactivated RuvC or NUC domain, wherein the RuvC or NUC domain has no nuclease activity anymore. The skilled person is well aware of techniques available in the art such as site-directed mutagenesis, PCR-mediated mutagenesis, and total gene synthesis that allow for inactivated nucleases such as inactivated RuvC or NUC domains. An example of a Cpf1 nickase with an inactive NUC domain is Cpf1 R1226A (see Gao et al. Cell Research (2016) 26:901-913, Yamano et al. Cell (2016) 165(4): 949-962). In this variant, there is an arginine to alanine (R1226A) conversion in the NUC-domain, which inactivates the NUC-domain.

The CRISPR-protein encoded by the coding RNA may be, or may be derived from, CRISPR-Casϕ, a nuclease that is about half the size of Cas9. CRISPR-Casϕ uses a single crRNA for targeting and cleaving the nucleic acid as is described e.g. in Pausch et al (CRISPR—Casϕ from huge phages is a hypercompact genome editor, Science (2020); 369(6501):333-337).

An active, partly inactive or dead CRISPR-protein encoded by the coding RNA may serve to guide a fused functional domain as detailed herein to a specific site in the DNA as determined by the guide RNA.

Hence, the CRISPR-protein may be fused to a functional domain. Optionally, such functional domain is for epigenetic modification, for example a histone modification domain. The domains for epigenetic modification can be selected from the group consisting of a methyltransferase, a demethylase, a deacetylase, a methylase, a deacetylase, a deoxygenase, a glycosylase and an acetylase (Cano-Rodriguez et al, Curr Genet Med Rep (2016) 4:170-179). The methyltransferase may be selected from the group consisting of G9a, Suv39h1, DNMT3, PRDM9 and Dot1L. The demethylase may be LSD1. The deacetylase may be SIRT6 or SIRT3. The methylase may be at least one of KYP, TgSET8 and NUE. The deacetylase may be selected from the group consisting of HDAC8, RPD3, Sir2a and Sin3a. The deoxygenase may be at least one of TET1, TET2 and TET3, preferably TET1cd (Gallego-Bartolome J et al, Proc Natl Acad Sci USA. (2018); 115(9):E2125-E2134). The glycosylase may be TDG. The acetylase may be p300.

Optionally, the functional domain is a deaminase, or functional fragment thereof, selected from the group consisting of an apolipoprotein B mRNA-editing complex (APOBEC) family deaminase, an activation-induced cytosine deaminase (AID), an ACF1/ASE deaminase, an adenine deaminase, and an ADAT family deaminase. Alternatively or in addition, the deaminase or functional fragment thereof may be ADAR1 or ADAR2, or a variant thereof.

The apolipoprotein B mRNA-editing complex (APOBEC) family of cytosine deaminase enzymes encompasses eleven proteins that serve to initiate mutagenesis in a controlled and beneficial manner. Preferably, the APOBEC deaminase is selected from the group consisting of APOBEC1, APOBEC2, APOBEC3A, APOBEC3B, APOBEC3C, APOBEC3D, APOBEC3F, APOBEC3G, APOBEC3H, APOBEC4 and Activation-induced (cytidine) deaminase. Preferably, the cytosine deaminase of the APOBEC family is activation-induced cytosine (or cytidine) deaminase (AID) or apolipoprotein B editing complex 3 (APOBEC3). Preferably, the deaminase domain fused to the CRISPR-protein an APOBEC1 family deaminase.

Another exemplary suitable type of deaminase domain that may be fused to the CRISPR-system nuclease is an adenine or adenosine deaminase, for example an ADAT family of adenine deaminase. Further, the adenine deaminase may be TadA or a variant thereof, preferably as described in Gaudelli et al., 2017 (Gaudelli et al. 2017 Nature 551: 464-471). Further, the CRISPR-system nuclease may be fused to an adenine deaminase domain, e.g. derived from ADAR1 or ADAR2. The deaminase domain of the present invention may comprise or consist of a whole deaminase protein or a fragment thereof which has catalytic activity. Preferably, the deaminase domain has deaminase activity. Optionally, the CRISPR-protein is further fused to an UDG inhibitor (UGI) domain.

The coding RNA of the invention may comprise two or more sequences encoding a CRISPR-protein. Optionally, the coding RNA may encode two times or more often the same CRISPR-protein. Alternatively or in addition, the coding RNA encodes two or more different CRISPR-proteins, such as, but not limited to a CRISPR-Cas9 and a CRISPR-Cpf1.

### A Guiding RNA

In the target cell, the coding RNA is translated, resulting in the expression of a CRISPR-protein. This CRISPR-protein is associated with a guide RNA, which guides the CRISPR-protein to a specific location in the genome of a plant cell to achieve a targeted genomic modification.

In an aspect, the invention therefore also pertains to a combination of a coding RNA as defined herein and one or more guide RNAs. The guide RNA may be comprised in a guiding RNA. A guiding RNA comprises a guide RNA and a mobile element. Optionally, the guiding RNA comprises two or more mobile elements and/or two or more guide RNAs. The invention therefore also pertains to a combination of a coding RNA as defined herein and one or more guiding RNAs.

The guide RNA directs the complex to a defined target site in a double-stranded nucleic acid molecule, also named the protospacer sequence. The guide RNA comprises a sequence for targeting the CRISPR-protein complex to a protospacer sequence that is preferably near, at or within a sequence of interest in the genome of the plant cell.

The guide RNA can be a single guide (sg)RNA molecule or the combination of a crRNA and a tracrRNA (e.g. for Cas9) as separate molecules or a crRNA molecule only (e.g. in case of Cpf1 and Casϕ). Preferably, the guide RNA comprises at least one of a single guide RNA (e.g. for Cas9) or a crRNA only (e.g. in case of Cpf1 and Casϕ).

The CRISPR-protein complex for use in the method of the invention may thus comprise a guide RNA, wherein the guide RNA comprises a combination of a crRNA and a tracrRNA, and wherein preferably the CRISPR-protein is Cas9. The crRNA and tracrRNA are preferably combined into a sgRNA (single guide RNA). Alternatively, the CRISPR-protein complex for use in the method of the invention may comprise a guide RNA, wherein the guide RNA comprises a crRNA, and wherein preferably the CRISPR protein is Cpf1 or Casϕ.

The guide RNA for use in a method of the invention may comprise a sequence that can hybridize to or near a sequence of interest, preferably a sequence of interest as defined herein. The guide RNA may comprise a nucleotide sequence that is fully complementary to a sequence in the sequence of interest i.e. the sequence of interest comprises a protospacer sequence. Alternatively or in addition, the guide RNA for use in the method of the invention may comprise a sequence that can hybridize to or near the complement of a sequence of interest.

The part of the crRNA that is complementary to the protospacer sequence is designed to have sufficient complementarity with the protospacer sequence to hybridize with the protospacer sequence and direct sequence-specific binding of a complexed CRISPR protein. The protospacer sequence is preferably adjacent to a protospacer adjacent motif (PAM) sequence, which PAM sequence may interact with the CRISPR protein of the RNA-guided CRISPR-protein complex. For instance, in case the CRISPR protein is S. pyogenes Cas9, the PAM sequence preferably is 5′-NGG-3′, wherein N can be any one of T, G, A or C.

The skilled person is capable of engineering the crRNA to target any desired sequence, preferably by engineering the sequence to be at least partly complementary to any desired protospacer sequence, in order to hybridize thereto. Preferably, the complementarity between part of a crRNA sequence and its corresponding protospacer sequence, when optimally aligned using a suitable alignment algorithm, is at least 70%, 80%, 85%, 90%, 91%, 92%, 93%, 94%, 95%, 96%, 97%, 98% or 100%. The part of the crRNA sequence that is complementary to the protospacer sequence may be at least about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some preferred embodiments, the sequence complementary to the target sequence is less than about 75, 50, 45, 40, 35, 30, 25, 20 nucleotides in length. Preferably, the length of the sequence complementary to the target sequence is at least 17 nucleotides. Preferably the complementary crRNA sequence is about 10-30 nucleotides in length, about 17-25 nucleotides in length or about 15-21 nucleotides in length. Preferably the part of the crRNA that is complementary to the protospacer sequence is 15, 16, 17, 18, 19, 20, 21, 22, 23, 24 or 25 nucleotides in length, preferably 20 or 21 nucleotides, preferably 20 nucleotides.

Molecules or sequences suitable as crRNA and tracrRNA are well known in the art (see e.g., WO2013142578 and Jinek et al., Science (2012) 337, 816-821). Preferably, the crRNA and tracrRNA are linked to together to form a single guide (sg)RNA. The crRNA and tracrRNA can be linked, preferably covalently linked, using any conventional method known in the art. Covalent linkage of the crRNA and tracrRNA is e.g. described in Jinek et al. (supra) and WO13/176772, which are incorporated herein by reference. The crRNA and tracrRNA can be covalently linked using e.g. linker nucleotides or via direct covalent linkage of the 3′ end of the crRNA and the 5′ end of the tracrRNA.

The guide RNA may be linked to a mobile element as defined herein, resulting in a guiding RNA. Preferably at least when the guide RNA is not part of an editing RNA as defined herein, the guide RNA is linked to a mobile element. A guiding RNA thus comprises two elements, a mobile element and a guide RNA. The mobile element may be directly linked to the guide RNA. Alternatively, there may be one or more nucleotides located in between the mobile element and the guide RNA, e.g. about 1-500, 5-400, 10-300, 15-200 or 20-100 nucleotides. The mobile element enables intercellular translocation of the guide RNA.

The mobile element of said guiding RNA may be the same element or a different element that is present in said coding RNA. Preferably, the mobile element present in said guiding RNA and the mobile element present in said coding RNA direct both RNA molecules to the same target tissue. Preferably, the mobile element present in said guiding RNA molecule and the mobile element present in said coding RNA molecule are the same, or substantially the same, mobile elements.

The guiding RNA may comprise 2, 3, 4, 5, 6, 7, 8, 9, 10 or more mobile elements. The mobile element, or elements, may be located upstream and/or downstream of the sgRNA. In addition or alternatively the mobile element, or elements, may be located upstream and/or downstream of the crRNA.

Optionally, the guiding RNA as defined herein may comprise more than one sgRNA and/or more than one crRNA, for example to target two or more different sequences of interest, or to target to or more locations of the same sequence of interest.

The guiding RNA may comprise two or more guide RNA sequences. The guiding RNA may comprise at least 1, 2, 3, 4, 5, 6, 7, 8, 9, 10 or more guide RNA sequences. Put differently, the guiding RNA may comprise at least 1, 2, 3, 4, 5, 6, 7, 8, 9, 10 or more single guide (sg)RNAs and/or at least 1, 2, 3, 4, 5, 6, 7, 8, 9, 10 or more crRNAs.

The different guide RNA sequences in the guiding RNA may direct the CRISPR protein to different locations in the same gene. Alternatively or in addition, the different guide sequences in the guiding RNA may direct the CRISPR protein to different genes in the plant cell.

There may be a sequence of at least 1, 2, 3, 4, 5, 6, 7, 8, 9, 10, 15, 20, 25 or more nucleotides that separate the different guide RNAs in the guiding RNA. Optionally, the different guide RNAs in the guiding RNA are separated by a cleavable spacer sequence, preferably a cleavable spacer sequence as defined herein. The guiding RNA may further comprise multiple mobile elements, preferably located in the guiding RNA such that after cleaving the spacer sequence, each cleaved RNA molecule comprises a guide RNA, in addition to a mobile element.

An editing RNA
The guide RNA and the coding RNA may be comprised in a single editing RNA, the editing RNA comprises at least the following elements:
i) A sequence encoding a CRISPR protein;
ii) A first mobile element; and
iii) A guide RNA.

The sequence encoding a CRISPR protein and the first mobile element may be part of the coding RNA as defined herein. Alternatively or in addition, the guide RNA may be part of the guiding RNA as defined herein. Preferably, The editing RNA therefore comprises at least the following two elements:
i) a coding RNA defined herein; and
ii) a guide RNA.
   Optionally, the editing RNA comprises the following two elements:
i) a coding RNA defined herein; and
ii) a guiding RNA as defined herein.
   The sequence encoding a CRISPR protein may be located 5′ of the guide RNA. Alternatively, the guide RNA may be located 5′ of the sequence encoding a CRISPR-protein. The sequence encoding a CRISPR protein may be located 5′ of the guiding RNA. Alternatively, the guiding RNA may be located 5′ of the sequence encoding a CRISPR-protein. As detailed herein, the guiding RNA may comprise multiple guide RNAs, optionally separated with cleavable spacer sequences and/or mobile elements. The sequence encoding the CRISPR protein is preferably located at the 5′ end or at the 3′ end of these multiple guide RNAs.

The editing RNA preferably comprises at least one or more mobile elements. These one or more mobile elements can be located at the 5′ end of the editing RNA, at the 3′ end of the editing RNA, in between the sequence encoding a CRISPR-protein and the guide RNA and/or in between the multiple guide RNAs within the guiding RNA.

The editing RNA may comprise at least two mobile elements, e.g. a first and a second mobile element. Preferably, at least one of the first and second mobile element is located at the 5′-end or at the 3′-end of the editing RNA. One of the mobile elements preferably directly flanks the sequence encoding a CRISPR-protein and one of the mobile elements preferably directly flanks the guide RNA. Hence one of the mobile elements is located in close to the 5′-end or 3′-end of the sequence encoding a CRISPR-protein and the other mobile element is located close to the 5′-end or 3′-end of the guide RNA.

The editing RNA may further comprise one or more cleavable spacer sequences. Preferably, cleavable spacer is located in between the sequence encoding a CRISPR protein and the guide RNA. Preferably, the spacer sequence is located in between the coding RNA and the guide RNA.

In case the editing RNA comprises a cleavable spacer sequence, the editing RNA also preferably comprises at least two mobile elements. Preferably, these at least two mobile elements translocate RNA molecules to the same target tissue. Preferably, the at least two mobile elements are the same, or substantially the same, mobile elements.

Preferably, the cleavable spacer sequence is located in the editing RNA such that cleavage results in separate RNA molecules, wherein each of the separated RNA molecules comprises a mobile element. As a non-limiting example, cleavage of the editing RNA results in a coding RNA molecule as defined herein and in a guiding RNA molecule as defined herein, wherein the guiding RNA molecule comprises a mobile element.

In case the editing RNA comprises a cleavable spacer sequence, the orientation of the different elements in the editing RNA may be as follows:
a) a first mobile element;
b) a sequence encoding a CRISPR protein;
c) a cleavable spacer;
d) a second mobile element;
e) a guide RNA.

The skilled person readily understands that alternative rearrangements in the editing RNA are equally suitable, such as, but not limited to:
b), a), c), d) and e);
a), b), c), e) and d);
b) a), c), e) and d);
a), e), c), d), b);
e), a), c), d) and b);
a), e), c), b) and d); and
e) a), c), b) and d).

The guide RNA and the mobile element together can also be annotated as a “guiding RNA” as defined herein. The skilled person understands that the guiding RNA may comprise one, two or more guide RNAs and further mobile elements and cleavable spacer sequences as detailed herein. The cleavable sequence in the editing RNA may thus be located in between the two or more guide RNAs of the guiding RNA.

The editing RNA may be cleaved in the first (production) cell and/or in the second (target) cell. The cleavable spacer sequence may be any sequence known to the skilled person suitable for cleaving an RNA molecule in a plant cell. Non-limiting examples of such cleavable spacer sequences include, but are not limited to a spacer having the following sequence:

(SEQ ID NO: 27)
UCGCGGCCGGGUACGUGUUGAGC

The cleavable sequence may be sequence having at least 85%, 90%, 95%, 96%, 97%, 98%, 99% or 100% sequence identity with SEQ ID NO: 27.

Alternatively, the cleavable spacer sequence can be any, preferably plant-derived, tRNA sequence. The cleavable tRNA sequence may be a naturally occurring sequence, or may be modified to comprise a cleavable sequence. The tRNA sequence may be a tRNA-like structure as defined herein. The cleavable tRNA sequence may be a cleavable mobile element as defined herein. The cleavable tRNA sequence may comprise a recognition site for cleavage by at least one of RNase P and RNase Z. the cleavable tRNA sequence preferably has a tRNA leader sequence at, or in close vicinity of, the 3′ and/or 5′-end, preferably at, or in close vicinity of the 5′-end. The tRNA leader sequence preferably comprises the sequence AACAAA, or a sequence that differs one or two nucleotides from said sequence. Suitable tRNA cleavable elements are e.g. described in Xie et al (“Boosting CRISPR/Cas9 multiplex editing capability with the endogenous tRNA-processing system”, 2015, Proc Natl Acad Sci USA; 112(11):3570-5), which is incorporated herein by reference.

### Chemical Modifications of the RNA(s)

The RNAs of the invention include at least one of a coding RNA, a guiding RNA and an editing RNA.

At least one of the RNAs of the invention may comprise or consist of non-modified or naturally occurring nucleotides. Optionally, all RNAs used in the method of the invention may comprise or consist of non-modified or naturally occurring nucleotides.

Alternatively or in addition, the at least one of the RNAs of the invention may comprise or consist of modified or non-naturally occurring nucleotides. Optionally, all RNAs of the invention may comprise or consist of modified or non-naturally occurring nucleotides. Such chemically modified nucleotides preferably protect the RNA(s) against degradation. Optionally, the at least one of the RNAs, i.e. at least one of the coding RNA, the guiding RNA and the editing RNA, comprises ribonucleotides and non-ribonucleotides. At least one of the RNAs may comprise one or more ribonucleotides and one or more deoxyribonucleotides.

Optionally, at least one of the RNAs, i.e. at least one of the coding RNA, the guiding RNA and the editing RNA, comprises one or more non-naturally occurring nucleotides or nucleotide analogues, such as a nucleotide with phosphorothioate linkage, a locked nucleic acid (LNA) nucleotides comprising a methylene bridge between the 2′ and 4′ carbons of the ribose ring, bridged nucleic acids (BNA), 2′-O-methyl analogues, 2′-deoxy analogues, 2′-fluoro analogues or combinations thereof. The modified nucleotides may comprise modified bases selected from the group consisting of, but not limited to, 2-aminopurine, 5-bromo-uridine, pseudouridine, inosine, and 7-methylguanosine.

At least one of the RNAs, i.e. at least one of the coding RNA, the guiding RNA and the editing RNA, may be chemically modified by incorporation of 2′-O-methyl (M), 2′-O-methyl 3′phosphorothioate (MS), 2′-O-methyl 3′thioPACE (phosphonoacetate) (MSP), or a combination thereof, at one or more terminal nucleotides. Such chemically modified RNAs can comprise increased stability and/or increased activity as compared to unmodified RNAs. (Hendel et al, 2015, Nat Biotechnol. 33(9); 985-989; Maruggi et al, Mol Ther, 2019 April 10; 27(4):757-772). In an embodiment of the invention, deoxyribonucleotides and/or nucleotide analogues can be incorporated in the engineered RNA structures.

### Plant

Any plant can be suitable for use in the invention. The invention is thus not limited to any specific plant, A preferred plant is a vascular plant or tracheophyte.
The plant for use in the invention can be a monocot or dicot. The plant can be a crop or a grain plant. A non-limiting example of a grain plant for use in the invention is cassava, corn, sorghum, soybean, wheat, oat or rice. A crop plant is a plant species which is cultivated and bred by humans. A crop plant may be cultivated for food purposes (e.g. field crops), or for ornamental purposes (e.g. production of flowers for cutting, grasses for lawns, etc.). A crop plant as defined herein also includes plants from which non-food products are harvested, such as oil for fuel, plastic polymers, pharmaceutical products, cork and the like.

The plant may also be a tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; plants of the genus Solanum, preferably Solanum lycopersicum). Preferably, the plant is a plant of the genus Solanum. A preferred plant for use in the invention is a Solanum lycopersicum plant.

In another preferred embodiment, the cell is obtainable from a plant selected from the group consisting of asparagus, barley, blackberry, blueberry, broccoli, cabbage, canola, carrot, cassava, cauliflower, chicory, cocoa, coffee, cotton, cucumber, eggplant, grape, hot pepper, lettuce, maize, melon, oilseed rape, pepper, potato, pumpkin, raspberry, rice, rye, sorghum, spinach, squash, strawberry, sugarcane, sugar beet, sunflower, sweet pepper, tobacco, tomato, water melon, wheat, and zucchini.

Preferably, the plant for use in the invention comprises one or more meristem tissues that are not or not efficiently accessible for viral delivery vectors. Preferably, the plant for use in the invention comprises one or more shoot apical meristem tissues that are not or not efficiently accessible for viral delivery vectors.

Preferably, the plant for use in the invention is not Nicotiana benthamiana, preferably not Nicotiana benthamiana GenBank accession PRJNA170566.

### Delivery Vectors

At least one of the coding RNA, the guiding RNA and the editing RNA as defined herein can be introduced into the phloem by direct injection of the RNA(s) into the vascular tissue of the plant. In this respect, the coding RNA, the guiding RNA and/or the editing RNA may comprise a chemical modification, e.g. to increase the stability of the RNA.

Alternatively or in addition, at least one of the coding RNA, the guiding RNA and the editing RNA as defined herein is introduced into a plant cell by introducing a vector expressing said RNA(s) into the plant cell. Hence in an aspect the invention pertains to a vector expressing an RNA of the invention. The invention thus relates to at least one of a vector expressing the coding RNA as defined herein, a vector expressing the guiding RNA as defined herein and a vector expressing an editing RNA as defined herein. A vector of the invention may express a combination of RNAs of the invention. For example, a vector may express a coding RNA as well as a guiding RNA.

A vector may be used to express the RNA of the invention in a plant host cell (the first, production, cell). The RNAs of the invention may thus be transcribed from an expression cassette comprised in the vector. The vector backbone may for example be a plasmid into which the expression cassette is integrated or, if a suitable transcription regulatory sequence is already present (for example a (inducible) promoter), only a desired nucleotide sequence (e.g. a sequence encoding one or more of the RNAs of the invention) is integrated downstream of the transcription regulatory sequence.

Suitable promoters, preferably suitable promoters for expression of at least one of the editing RNA, guide RNA and coding RNA include, but are not limited to, viral native coat protein promoters. Such native coat protein promoter for use in the invention may for example be derived from the tobacco rattle virus (as described e.g. in Deng X et al, 2013, Modification of Tobacco rattle virus RNA1 to Serve as a VIGS Vector Reveals That the 29K Movement Protein Is an RNA Silencing Suppressor of the Virus, Mol Plant-Microbe Interact. doi: 10.1094/MPMI-12-12-0280-R), the Pea early-browning virus (as described e.g. in MacFarlane SA and Popovich AH (2000) Efficient expression of foreign proteins in roots from tobravirus vectors. Virology. doi: 10.1006/viro.1999.0098), any one of the tobacco rattle virus strains TCM, PLB, PSG and PRV (as described e.g. in Goulden M G et al, 1990, The complete nucleotide sequence of PEBV RNA2 reveals the presence of a novel open reading frame and provides insights into the structure of tobraviral subgenomic promoters. Nucleic Acids Res. doi: 10.1093/nar/18.15.4507). The promoter may be a PEBV promoter, a U6 promoter or a pol III promoter.

The vector of the invention may comprise further genetic elements to facilitate their use in molecular cloning, such as e.g. selectable markers, multiple cloning sites and the like. The vector backbone may for example be a binary or superbinary vector (see e.g. U.S. Pat. No. 5,591,616, US 2002138879 and WO 95/06722), a co-integrate vector or a T-DNA vector, as known in the art.

Vectors according to the invention are particularly suitable for introducing the expression of the RNAs of the invention into a plant cell. A preferred expression vector is a naked DNA, a DNA complex or a viral vector.

A preferred naked DNA is a linear or circular nucleic acid molecule, e.g. a plasmid. A plasmid refers to a circular double stranded DNA loop into which additional DNA segments can be inserted, such as by standard molecular cloning techniques. A DNA complex can be a DNA molecule coupled to any carrier suitable for delivery of the DNA into the cell. A preferred carrier is selected from the group consisting of a lipoplex, a liposome, a polymersome, a polyplex, PEG, a dendrimer, an inorganic nanoparticle, a virosome and cell-penetrating peptides.

The vector is preferably a viral vector. The viral vector van be an DNA virus or an RNA virus. The viral vector may be, or may be based on, a Tobamovirus, a, Tobravirus, a, Potexvirus a Geminivirus, an Alfamovirus, a Cucumovirus, a Potyvirus, a Tombusvirus, a hordeivirus, or a Nucleorhabdovirus.

The Tobamovirus viral vector may be at least one of a Tobacco Mosaic Virus (TMV) and a Sun Hemp Mosaic Virus (SHMV). The Tobravirus viral vector may be a Tobacco Rattle Virus (TRV). The Potex virus viral vector may be at least one of Potatovirus X (PVX) and the papaya mosaic potexvirus (PapMV). The Geminivirus viral vector may be a Comovirus Cowpea mosaic virus (CPMV). Further examples of suitable Geminivirus viral vectors may include the cabbage leaf curl virus, tomato golden mosaic virus, bean yellow dwarf virus, African cassava mosaic virus, wheat dwarf virus, miscanthus streak mastrevirus, tobacco yellow dwarf virus, tomato yellow leaf curl virus, bean golden mosaic virus, beet curly top virus, maize streak virus, and tomato pseudo-curly top virus. The Alfamovirus may an alfalfa mosaic virus (AMV). The Cucumovirus may be a cucumber mosaic virus (CMV). The Potyvirus may be a plum pox virus (PPV). The Tombusvirus may be a tomato bushy stunt virus (TBSV). The hordeivirus may be a barley stripe mosaic virus. The Nucleorhabdovirus may be a Sonchus Yellow Net Virus (SYNV) (see e.g. Hefferon K, Plant Virus Expression Vectors: A Powerhouse for Global Health, Biomedicines. 2017, 5(3): 44 and Lico et al, Viral vectors for production of recombinant proteins in plants, J Cell Physiol, 2008; 216(2):366-77).

Preferably, the viral vector is selected from the group consisting of a Tobacco Rattle Virus (TRV), Tobacco Mosaic Virus (TMV), a Sonchus Yellow Net Virus (SYNV) and Potato Virus X (PVX). Preferably, the viral vector is at least one of a Tobacco Rattle Virus (TRV), a Tobacco Mosaic Virus (TMV) and a Sonchus Yellow Net Virus (SYNV).

The vector for use in the invention preferably does not express a functional coat protein. The sequence encoding the coat protein may comprise a mutation to abolish the expression of a functional coat protein. The mutation may be at least one of a deletion, an addition, a substitution or a combination thereof, preferably resulting in an early stop codon. Preferably at least part of the sequence encoding the coat protein is deleted. Preferably, at least 2%, 5%, 10%, 20%, 30%, 40%, 50%, 60%, 70%, 80%, 90% or 100% of the sequence encoding the viral coat protein is deleted from the viral genome.

Thus the viral vector expressing the RNA(s) of the invention may comprise a deletion of one or more genes, e.g. to increase the packaging capacity of the virus, to improve genome stability and/or to increase gene expression. In a preferred embodiment, the virus comprises a mutation, preferably a deletion, of at least part of a gene encoding the coat protein (CP). A preferred viral vector comprising a mutation, preferably a deletion, of at least part of a sequence encoding the coat protein is a Tobamovirus virus or a Tobravirus virus. Preferably the viral vector comprising a deletion of a coat protein is a Tobamovirus, preferably the Tobacco Mosaic Virus (TMV). A preferred viral vector is the TMV RNA-based overexpression vector (TRBO), e.g. as described in Lindbo (TRBO: A High-Efficiency Tobacco Mosaic Virus RNA-Based Overexpression Vector, Plant Physiol, 2007; 145(4):1232-40).

Infection of a plant cell with e.g. an TRBO or any other suitable vector preferably results in high level expression of the RNAs of the invention in the first, production, plant cell. High level expression of an RNA of the invention may for example be achieved by deleting the viral coat protein and/or the use of a strong promoter. The viral vector may be a self-replicating RNA as e.g. described in WO2018/226972, which is incorporated herein by reference.

The vector, preferably the viral vector, may be comprised in an Agrobacterium to initially introduce the viral vector into a cell of the plant. After infection, the viral vector is expressed from the Agrobacterium in the plant cell. The viral vector may express one or more RNAs of the invention and additionally may replicate and infect surrounding plant cells. The viral vector may be modified, e.g. by deletion of the coat protein, which prevents systemic spread of the virus.

In an aspect, the invention therefore pertains to an Agrobacterium, wherein the Agrobacterium expresses a vector as defined herein. Preferably the Agrobacterium is Agrobacterium tumefaciens.

In a further aspect, the invention concerns a method for producing a plant cell having a targeted genomic modification. Preferably, the plant cell is a meristem cell. The plant cell is preferably a shoot, a root or a leaf cell. Preferably the plant cell for use in the method of the invention comprises a cell wall. Preferably, the plant cell is not a plant cell protoplast. The method preferably comprises the steps of
i) providing a plant; and
ii) expressing in a cell of the plant a coding RNA as defined herein and a guide RNA.
   Optionally the guide RNA is comprised in a guiding RNA. Optionally, the coding RNA and guide RNA, or guiding RNA, are comprised in an editing RNA. The expressed RNA(s) may subsequently translocate to the second “target” cell. Preferably, the coding RNA and guide RNA translocate to a meristem cell. Preferably, the coding RNA and guide RNA translocate to a shoot apical meristem cell.

Preferably, the coding RNA is comprised within an editing RNA. Preferably, the coding RNA and a guide RNA are comprised within an editing RNA as defined herein. Alternatively, the coding RNA and the guide RNA are separate entities. In this embodiment, the guide RNA is preferably comprised in an guiding RNA. In an embodiment, the coding RNA and guiding RNA are separate entities.

Preferably in the second “target” cell, a CRISPR-nuclease is expressed from the coding RNA and the guide RNA directs the expressed CRISPR-nuclease to a location in the genome to generate a targeted genomic modification in the target cell. Preferably the target cell is a meristem cell. Hence preferably in the meristem cell, a CRISPR-nuclease is expressed from the coding RNA and the guide RNA directs the expressed CRISPR-nuclease to a location in the genome to generate a targeted genomic modification in the meristem cell.

Preferably, the coding RNA and the guiding RNA, are expressed in the (first or “target”) cell as separate entities or within an editing RNA by transfecting the plant cell with a vector as described herein. Preferably, the plant cell is transfected with a vector expressing an editing RNA as described herein and/or a combination of a first and a second vector, wherein the first vector expresses a coding RNA as described herein and the second vector expresses a guiding RNA as defined herein.

The vector may be introduced into a plant cell by transfecting the cell with a Agrobacterium comprising a vector as defined herein.

The cell comprising the targeted genomic modification may subsequently be developed into a new plant comprising the genomic modification. The method of the invention may thus further comprise a step of developing a plant from the cell, preferably from the meristem cell, comprising the targeted genomic modification.

Developing a plant from the plant cell comprising the targeted genomic modification can be done using any conventional method known in the art. As a non-limiting example, the cell comprising the targeted genomic modification may develop into pollen and/or egg cells, followed by self-pollination or the pollination of a second plant comprising cells having the same or a different targeted genomic modification

Alternatively, a meristem cell having a targeted genomic modification may develop into a plant by means of regeneration e.g. after decapitation. As a non-limiting example, one or more plant cells of a plant, preferably one or more cells of a cotyledon, may be transfected using a method as defined herein, followed by decapitation of preferably the shoot apical meristem. The subsequently newly formed meristem cells (i.e. the “second” or “target” cells) may comprise the targeted genomic modification and these newly formed meristem cells may regenerate into a plant comprising cells having the targeted genomic modification. Alternatively, the plant may first be decapitated, followed by transfection of the first “producer” cells and subsequent genomic modification of the newly formed meristem cell (the “second” cell). The newly formed meristem cell may regenerate into a plant comprising cells having the targeted genomic modification.

The invention further pertains a plant cell or a plant obtainable by the method of the invention. Preferably, the plant cell is a meristem cell. Preferably, the plant cell and/or plant comprises a targeted genomic modification. The plant of the invention preferably differs at least from a plant occurring in nature, in that it contains at least one mutation in one gene of interest. Preferably, the plant of the invention is not, or is not exclusively, obtained by an essentially biological process.

All or substantially all cells of the plant may comprise the targeted genomic modification. Alternatively, only part of the plant comprises the targeted genomic modification. Preferably, one or more meristem cells in the plant comprises the targeted modification.

The present invention has been described above with reference to a number of exemplary embodiments. Modifications and alternative implementations of some parts or elements are possible, and are included in the scope of protection as defined in the appended claims.

### FIGURE LEGEND

FIG. 1: A schematic overview of the vector containing the Cas9-GFP fusion with the FT sequence (A) and without the FT sequence (B).

FIG. 2: The average expression (in arbitrary units relative to expression of N. benthamiana tubulin mRNA), as determined by qPCR, of Cas9 and GFP in the shoot apex of plants infiltrated in the leaves with a Cas9-GFP construct with or without a FT signal.

### EXAMPLES

### Example 1

We explored the use of Tobacco Rattle Virus (TRV) for the transient expression of CAS9 and guide RNA sequences in plants, with the aim of inducing somatic edits. TRV infection occurs through the transient expression of two vectors: TRV1 and TRV2. Sequences can be cloned in TRV2, and through the use of subgenomic promoters, expression of these sequences occurs in the plant. The goal of this experiment was to detect somatic edits in tomato cotyledons through infection with a TRV2 plasmid expressing two (pKG11581) or three (pKG11580) guideRNAs as well as CAS9.

### Material and Methods:

Two TRV2 plasmids were made through the introduction of two sequences. The first sequence contained either two (pKG11581) or three (pKG11580) guideRNAs. The second sequence is for expression of NLS:Sp-CAS9 and this sequence was cloned in the TRV2 plasmid, thereby replacing the sequence encoding the TRV coat protein. Seedlings (several days post germination) of tomato cv. Moneyberg (De Ruiter Seeds CV, The Netherlands) were infiltrated in both cotyledons with a mixture of Agrobacterium samples containing TRV1:TRV2 (pKG11580 or pKG11581) in a 1:1 ratio. 2 days after infiltration, petiole samples were taken to detect CAS9 expression. 7 days after infiltration, cotyledon samples were taken for the detection of edits made by the guide RNAs targeting two separate genes.

### Results:

2 days after infiltration, CAS9 was detected through Q-PCR. 7 days after infiltration, one cotyledon per seedling was harvested and frozen. DNA was isolated, purified and PCR was performed to detect edits in the target sequences. Edits were not found in the control samples. In the transfected cotyledons, various edits were detected. We therefore conclude that The CAS9 is expressed, translated and functional and edits can be made with the sequences of CAS9 and guideRNAs as a cargo in the TRV virus.
Hence, the TRV2 plasmid can be considered as usable for the delivery of mobile RNAs. These mobile RNAs could then generate a CAS9 editing complex in meristem cells.

### Example 2

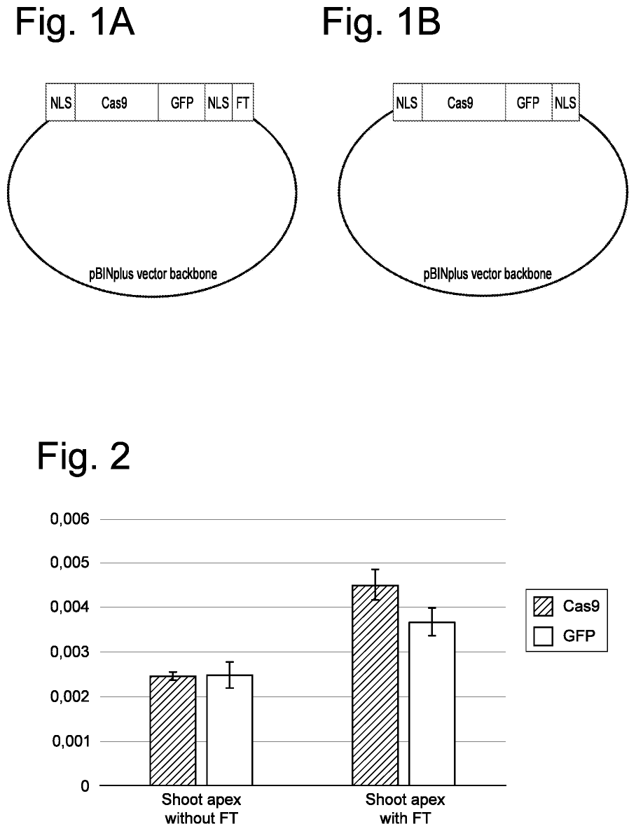

The influence of an exemplary mobile element (FT) on the mobility of a CAS9-GFP fusion mRNA was tested. N. benthaminana (Herbalistics, Australia) was grown for 5 weeks at 16 hours light (22° C.), 8 hours dark (20° C.) and 70% relative humidity. Five individual plants were leaf infiltrated with Agrobacterium tumefaciens, carrying a pBINplus vector, containing a CAS9-GFP-NLS insert (FIG. 1B). Another 5 individual plants were leaf infiltrated with A. tumefaciens, carrying a pBINplus vector, containing a CAS9-GFP-NLS-FT insert (FIG. 1A), containing the FT mobile sequence. Three days after infiltration, 2 mm long shoot apex samples without any discernible leaves were collected per plant. RNA was extracted with the Maxwell® RSC Plant RNA Kit (Promega, AS1500) on the Maxwell RSC instrument (Promega, AS4500). RNA was measured and 1 μg of RNA per sample was synthesized into cDNA with the QuantiTect® Reverse Transcription kit (Qiagen, order number 205311) according to the manufacturer's instructions. Quantitative PCR measurement for detecting CAS9 and GFP transcripts, and N. benthamiana tubulin mRNA as a reference, was done for all samples (for a list of primers see table 2).

The levels of mRNA were expressed in arbitrary units relative to expression of N. benthamiana tubulin mRNA. The average level of CAS9 and GFP transcripts was higher in the shoots of plants infiltrated with the CAS9-GFP construct containing the FT signal than in plants infiltrated with the construct lacking the FT signal (FIG. 2). To conclude, the FT sequence positively impacts the quantity of CAS9 transcripts in the shoot apices of plants that were leaf infiltrated with the pBINplus vector with CAS9-GFP-NLS-FT insert.

In a related example, wherein Solanum lycopersicum plants were leaf infiltrated with the same A. tumefaciens carrying the pBINplus vector with the CAS9-GFP-NLS-FT construct as in the present example, qPCR was performed on cDNA of the full length CAS9-GFP transcript. This was achieved by using CAS9 and GFP specific primers as shown in table 3. This resulted in a fragment of 3881 bp, covering 78% of the total transcript length. This transcript was detected in both the infiltrated leaves as well as the SAM, confirming that an intact transcript reaches the SAM, when applied into the leaves.

TABLE 2
The forward and reverse qPCR primer sequences
used to detect mRNA containing the GFP, CAS9
and N. benthamiana tubulin respectively.

| | | SEQ |
|---|---|---|
| | | ID |
| qPCR primers | Sequence | NO |
| GFP F | GGTCTTGTAGTTGCCGTCGT | 29 |
| GFP R | TCGTGACCACCCTGACCTAC | 30 |
| CAS9 F | AAGAGGCAGCTTGTGGAAAC | 31 |
| CAS9 R | ACACGAGCTTTGACTTGAGG | 32 |
| Tobacco a tubulin F | CAAGTGTTGCTGAGGTCTTCT | 33 |
| Tobacco a tubulin R | AGAACTCTCCTTCCTCCATACC | 34 |

TABLE 3
The forward and reverse qPCR primer
sequences used to detect mRNA con-
taining the full length CAS9-GFP
transcript and the EXP reference
transcript in the SAM of S.
lycopersicum.

| | | SEQ |
|---|---|---|
| | | ID |
| qPCR primer | Sequence | NO |
| CAS9 F | AGTTGTTCATCCAGCTCGTG | 35 |
| GFP R | GGTCTTGTAGTTGCCGTCGT | 36 |
| EXP reference F | GCTAAGAACGCTGGACCTAATG | 37 |
| EXP reference R | TGGGTGTGCCTTTCTGAATG | 38 |

1. A vector expressing a coding RNA, comprising a sequence encoding a CRISPR-nuclease and a first mobile element, wherein the mobile element enables intercellular translocation of the coding RNA.

2. The vector according to claim 1, wherein the CRISPR-nuclease comprises a nuclear localization signal.

3. The vector according to claim 1, where in the vector is a viral vector.

4. The vector according to claim 1, wherein the vector is:
a) a virus, that:
(i) does not express a functional coat protein; and/or
(ii) is a Tobacco Rattle Virus (TRV), a Tobacco Mosaic Virus (TMV) or a Sonchus yellow net virus (SYNV), a tobacco mosaic virus RNA-based overexpression vector (TRBO);
b) a naked DNA; or
c) a DNA molecule coupled to a carrier.

5. The vector according to claim 4, wherein the virus has a deletion in a sequence encoding the coat protein.

6. The vector according to claim 4, wherein the naked DNA is a circular nucleic acid molecule.

7. The vector according to claim 4, wherein the carrier is selected from the group consisting of a lipoplex, a liposome, a polymersome, a polyplex, PEG, a dendrimer, an inorganic nanoparticle, a virosome and cell-penetrating peptides

8. The vector according to claim 1, wherein the mobile element enables intercellular translocation to a meristem cell.

9. the vector according to claim 1, wherein the mobile element is a transfer-RNA (tRNA), a gene transcript, or both.

10. The vector according to claim 9, wherein the tRNA is at least one of a methionine, glycine, threonine-, arginine-, lysine- and glutamine-tRNA.

11. The vector according to claim 9, wherein the gene transcript is selected from the group consisting of FT, GAI, SP2G, SP3D, SP5G, SP9D, CEN-like protein 1, protein MOTHER of FT and TF 1, Flowering locus T-a, Flowering locus T-b, PP16-1, GAIP, SCARECROW-LIKE (SCL14P), SHOOT MERISTEMLESS (STMP), ETHYLENE RESPONSE FACTOR (ERFP) and Myb (MybP), wherein optionally the FT gene transcript is a mutant FT and/or truncated FT.

12. A vector according to claim 1, wherein the vector further comprises a guide RNA and optionally a second mobile element enabling intercellular translocation of the guide RNA.

13. An editing RNA comprising the coding RNA comprising (i) a sequence encoding a CRISPR-nuclease and a first mobile element, wherein the mobile element enables intercellular translocation of the coding RNA, (ii) a guide RNA, and optionally (iii) a second mobile element enabling intercellular translocation of the guide RNA.

14. The editing RNA according to claim 13, further comprising a cleavable spacer sequence located in between the coding RNA and the guide RNA.

15. The editing RNA according to claim 13, wherein the first and/or second mobile element is located at the 5′-end or at the 3′-end of the editing RNA.

16. The editing RNA according to claim 13, wherein the editing RNA comprises two or more guide RNAs.

17. The editing RNA according to claim 16, wherein the two or more guide RNAs direct the CRISPR-nuclease to the same gene.

18. A vector expressing an editing RNA according to claim 7.

19. An agrobacterium expressing the vector according to claim 1.

20. An agrobacterium expressing the RNA according to claim 13.

21. A method for producing a meristem cell having a targeted genomic modification, wherein the method comprises:
(i) providing a plant; and
(ii) expressing in a cell of the plant a coding RNA according to claim 1, a guide RNA, and optionally
(iii) a second mobile element enabling intercellular translocation of the guide RNA,
wherein the coding RNA and guide RNA translocate to a meristem cell,
wherein the coding RNA and the guide RNA are comprised within an editing RNA and/or wherein the guide RNA is linked to the second mobile element, and
wherein in the meristem cell a CRISPR-nuclease is expressed from the coding RNA and wherein the guide RNA directs the expressed CRISPR-nuclease to a location in the genome to generate a targeted genomic modification in the meristem cell.

22. The method according to claim 21, wherein the coding RNA and guide RNA are expressed by transfecting the plant cell with at least one of:
(i) a vector, comprising a sequence encoding a CRISPR-nuclease and a first mobile element, wherein the mobile element enables intercellular translocation of the coding RNA; and
(ii) an agrobacterium comprising the vector.

23. A meristem cell having a targeted genomic modification wherein the cell is obtainable by the method according to claim 21.


SEQUENCE LISTING (38 sequences)

SEQ ID NO: 1 - DNA, 975 bp, Arabidopsis thaliana
  source 1..975: mol_type = other DNA
agttaatgcaaatccgaaacagtataaatatgtgtagagggttcatgcctatgatacaaattaaagaagcagaaacaaaaacaagtaaaacagaaacaatcaacacagagaaaccacctgtttgttcaagatcaaagatgtctataaatataagagaccctcttatagtaagcagagttgttggagacgttcttgatccgtttaatagatcaatcactctaaaggttacttatggccaaagagaggtgactaatggcttggatctaaggccttctcaggttcaaaacaagccaagagttgagattggtggagaagacctcaggaacttctatactttggttatggtggatccagatgttccaagtcctagcaaccctcacctccgagaatatctccattggttggtgactgatatccctgctacaactggaacaacctttggcaatgagattgtgtgttacgaaaatccaagtcccactgcaggaattcatcgtgtcgtgtttatattgtttcgacagcttggcaggcaaacagtgtatgcaccagggtggcgccagaacttcaacactcgcgagtttgctgagatctacaatctcggccttcccgtggccgcagttttctacaattgtcagagggagagtggctgcggaggaagaagactttagatggcttcttcctttataaccaattgatattgcatactctgatgagatttatgcatctatagtattttaatttaataaccattttatgatacgagtaacgaacggtgatgatgcctatagtagttcaatatataagtgtgtaataaaaatgagagggggaggaaaatgagagtgttttacttatatagtgtgtgatgcgataattatattaatctacatgaaatgaagtgttatatttatactttacgtgtattcatttcttttcgatgcaaaaatcaggcagtgggaagaatctgctgttttacttttg

SEQ ID NO: 2 - DNA, 2376 bp, Cucurbita maxima
  source 1..2376: mol_type = other DNA
gggcagaacgggccgcgagaagctctgattcaatcattcagaagatgcgggttcacaccattttcctttgattctctctccttcttcccccatttttctacccaatctccaccgtctaaacccaacccttttttcctcctccgccaccgccgccgtttctacacacgatgaagagggagcatcactatcttcatcctcgtccggagccgccttccgtggctactggctctaatcgggagagttatttgaacactggtaaggctaagctctgggaggaggaggtacagctcgacggaggaatggatgagcttcttgctgttttaggttacaaggttaagtcgtcggatatggcggaagttgcacagaagctcgaacagcttgaagaagctatgtgtcaagttcaggatactggcctttcgcatctcgctttcgacactgttcattataatccctctgatctgtcgacttgggttgaaagtatgctcactgagctccatccgccccctacctcgcatctggacgattcatcgtttttagctccggcggaatcctccaccatcgctaacgttgattatgaacctcaactgcaaaccagcagcaggattttcgaggagtcttctagttcggattatgaccttaaagctattacggatagcgcgatttattcaccgagagagagtaagcgtttgaaagcctccgagtcggacacggatgtgttctccacctccgcgattggggcttctaattttgcaactcgcccagttgttctcgtcgattcgcaggagaacggaattcaactggttcatgctctgatggtttgtgctgaagccgtacagcagaataatctgaatctagcggaggctctggttaagcgaatcgattacttagcggtttctcaagccggagcgatgaggaaagtcgccacgttcttcgccgaagcattagcacgccgaatctacaggctctgccctgagaatcccctcgatcgttcagtgctcgatatgcttcagatgcatttctacgagagctgtccctatctgaaattcgcgcatttcaccgcgaatcaagcgattctcgaagccttcgaagggaagaagcgtgttcacgtcatcgatttctcgatgaaccaggggatgcagtggccggctttgattcaagccctagctctacgaccgagcggtcctcctaccttccgactcaccggaatcggccctccggcgccggataactccgattacctccaagatgtgggctggaagctcgccaaattcgctgaaaccctccatgtggagttcgaatacagaggatttgtagcgaacagtttggcagatctggacgcatcgatgctggagctccggccgagcgaggtggagtcggtggtagtgaactcggtgttcgagctgcatcagctactggctcggccgggggcaattgaaaaggttctgtcggtggtgaagcagatgaagccggagatcgtgacggtggtggagcaggaagcgaaccacaacggtccggttttcgtggaacgattcactgagtcactccattactactcgacgctgtttgactcgttggagtgctcccccaacagccaagacaagatgatgtcggaaatgtaccttgggaaacaaatctgcaatgtggtagcttgtgaaggtgccgaccgagtggaacgacacgaaaccctgactcagtggcgcactcggttatcctccgccgggttcgatcccatccacctcggctcaaacgcattcaagcaagcgagtatcctcctcgccctgtttggcagcggcgaagggtaccgggtagaagagaacgagggatcactaatgctgggatggcacacccgcccccttatcgccacctccgcctggaaacccggcaacaacccggtggtcgctcactgagtcaattccactcagtggaccgagtcaggtcaagaatgaaacaggactgactgtcagcttgacagagtgacccaccactcgcaaatgtggccatcacttttgcctgtcatcctgacccttcggcttcccgcccttcgtcttagtttggtgggtatcatatgaagggctcttctgtagttcttctgagtggtaaatttcggaagaagaaaaaggatttatgtatttatgtttattgaattaagtagtaaataaatgtggaaatgaattaattagattggtgggaataaaaagaaaaggagaaggaagaacaagagggtgttttggattagttagttagtggttcaatgaatgatgtgagcttttctttctttgtttcaaacacagtttcctgccaactcatccacttttttcagtgttttcaaagcatcttcaccacttacgtacttacccaaatgggggaagaattgaa

SEQ ID NO: 3 - DNA, 839 bp, Solanum lycopersicum
  source 1..839: mol_type = other DNA
ctgagacaagtcaccattaacatgacgtggaaattttctaaacacgtgtactctctttgtccatttcaacaaatttgcttcagtcacatgaaaaaagaaatataaattgatcagttttacaaatcaatcaattaatggcaaatatggaaacttcggcgaggtcggtagatcctctagtggtcggaaaggtgatcggagatgttttggacatgtttgttccggtggttgactttacggtggagtatgcttccaaacaaatatcgaataatggagttgaaattaagccagcagaagctgcacaaaagcctagagttcatataaagggttctcttcattctaataatctttacacccttgttatggctgatccagatgctcctagtccaagtgaaccgacttttagagaatggctccattggattgtcacggatattcctgagggaggtgatgcctctcaagggagggagatggtggaatatatgggaccgaagccaccggcagggatacatcgatacgtgttcactctgtttaggcagaaggaggcggagcaagtgccccataaaccaccacaagggcgatccaatttcaagactcgacagtttgcaagtgacaatggacttgatcttcctgtggctgcactgtactttaattcacaaaaggaacatgctgctcatcactagctctttacaatgtgtgctgctatatatagcaatgtaattactaccttatgcttgttagctagtattgcaagtgctttgcatatatagagttatgtatacttgtgtttatgtgaggtgcattatatttataagaatatgtgcatgtgctgtttgatatca

SEQ ID NO: 4 - DNA, 534 bp, Solanum lycopersicum
  source 1..534: mol_type = other DNA
atgcctagagaacgtgatcctcttgttgttggtcgtgtggtaggggatgtattggaccctttcacaagaactattggcctaagagttatatatagagatagagaagttaataatggatgcgagcttaggccttcccaagttattaaccagccaagggttgaagttggaggagatgacctacgtacctttttcactttggttatggtggaccctgatgctccaagtccgagtgatccaaatctgagagaataccttcactggttggtcaccgatattccagctaccacaggttcaagttttgggcaagaaatagtgagctatgaaagtccaagaccatcaatgggaatacatcgatttgtatttgtattattcagacaattaggtcggcaaacagtgtatgctccaggatggcgtcagaatttcaacacaagagattttgcagaactttataatcttggtttacctgttgctgctgtctattttaattgtcaaagagagagtggcagtggtggacgtagaagatctgctgattga

SEQ ID NO: 5 - DNA, 891 bp, Solanum lycopersicum
  source 1..891: mol_type = other DNA
atgcctagagatcctttaatagtttctggagttgttggagatgttgttgatccattcacaagatgtgtagactttggtgtggtttacaacaatagggtggtctataatggatgttccttgaggccttcacaagttgtcaatcaacctagggttgacattgatggagacgatcttcgtactttttacactctgattatggtggatcctgatgctccaaaccctagcaacccaaacctgagggaatatttgcactggttggtcacagatatcccagcagccacaggagcaacctttggcaatgaagtcgtgggctacgagagcccacgaccctcaatgggaatccatcgttatattttcgtgttgtatcgacaattgggctgcgatgccatcgatgcaccggacataatcgattctagacaaaatttcaacacaagagactttgctaggtttcacaatctaggtttgcctgttgctgctgtttacttcaattgcaatagggaaggtggtaccggtggtcgtcgcctataaatcacccccctctcctcggggtgcgatccgttctcgaactctgtgtcaatgtcagatgttttgtgtaacggattttttgtttgatagtcactcagctaaattgcttattaactcagaaagtcatttttctttttatcgaagaaaattgaaattatgagataatagctattatagttgagtgataatctgaaaaaaaaaatcaaccataatatatattatatattatatatatacccaaaaaataaaagtcttgtttggtatatattgatatatatgtatatgtgttttatattttattactgaattttaatttgtatgtatacgagtctacatgtaaaagtgtggatatatttactatatcta

SEQ ID NO: 6 - DNA, 525 bp, Solanum lycopersicum
  source 1..525: mol_type = other DNA
atgcaaagagaaagagatacattgagacttgctagagtgataggggatgttttggatccattcacaaagtctattaacctaagagttgtttataacaataaggagattagaaatggttgtgatttgaggccttctatggttgtcaaccaacctagggttgaagttggaggagacgattttcagactttttacactctggttatggtggatcctgatgctccaactcccagtaatccttgtcacaaggactatttgcactggttggtcactaacattccagcaagcacaggagtaacctttggaaatgaagttgtatcttatgagtgtccaagacctacaatgggaatacatcgacttgttctagttttatttcgtcaattgcgtcgagaaatcatctatgctcccgaaaatcgccaaaatttcgatacgagggaatttgcaaagctctataattttggattaccagtagctgcggtttatttcaattgtcaaagagaaaatggtactggtggtcgtagaatataa

SEQ ID NO: 7 - DNA, 925 bp, Nicotiana sylvestris
  source 1..925: mol_type = other DNA
ttctacttcaataattagattaccctagctagctcgccaaaaagaagaagaaacaaaagggtgaagatggcttctagggttgttgaaccacttgttgttgcacgagtgataggagaagtagtagacagtttcaacccaagtgtgaaactgaatgtgatatacaacgggagcaaacaagtatttaatgggcatgaactcatgcctgctgtcattgctgctaaacctcgtgtcgagattggtggcgaagacatgcgatctgcttacacccttatcatgaccgacccagatgttccaggtcctagtgatccttacttgagggaacacctccactggattgttacagatattcctggttccactgattcctcgttcggaagggaaatagtgagctacgagagcccaaagccagtgattgggattcaccgatatgtgttactattgtataaacaaagcggaaggcaaacagtgaaaccagcagcaacaagagaccatttcaacactcgaaggtatacggctgagaatggattgggaagcccagttgccgctgtctacttcaatgcccagagagaaactgctgccagaagaagatgaattactacatcataatataaacacgcaaataatcacttttagcttaattaggctgttaatgagctggaagtagtaactcctttaatgtactactctatctgtctattaaaacacgtgatttaaataaggtctaagtatatagtacaactttgtacttaccagaaaaatagctaggtgaattgatcaagaatagtgtcaatcttcatgcatcatgcagattctgagctttcccaccaattaggttgcccctgtgtttcatttgtttgtgagttctacttgtactttacttcatattagttgtttgatgtattaaattgagataaaatgcatatat

SEQ ID NO: 8 - DNA, 763 bp, Nicotiana sylvestris
  source 1..763: mol_type = other DNA
taaacccagtacctcaaattagtgttatatccatttttgcttcccaacaaacatatatttccattttaatggctgcaaaagttgatccgttggtggttggaagagtaattggggatgtagtggacatgttcgtgcccagtgtcactatgtctgtgcactttggcaacaaacatgttacaaatggttgcgatatcaaaccctctattgcggctgatcctccaaaaatcaccgtgggtggccatgctgctgacctttacactctggttatgacggatccagatgctcccagtccgagtgaaccaaatatgcgagagtgggtgcattggatcgtgacagacataccagggtgcagtaacgtcgctcgagggaaggaggtattggggtatgtggggccacgtccgccggtggggattcacaggtacatactggtacttttccgacagaaggcgccaatgccgggaattttgcagccgccactagctaggtctcatttctgcactcgggcgtttgcgcatcagcttgatctcggcgtccctgttgccactgtttatttcaatgcccataaggagccagcaaataggaagcgctgagcgagctgctttcactatcagtgaattttattttgttgtctgtatttagtttttactactagtatattctggcacttattattagccagcttattactctatgtttgcaacgtaaagtatgatttttatgttgctagatgctgctgtattgttatgtacctatattccgcgca

SEQ ID NO: 9 - DNA, 534 bp, Nicotiana sylvestris
  source 1..534: mol_type = other DNA
atgttaagagcaaatcctttagtagtatctggtgtaataggagatgtattggatccatttacaaagtctgtagactttgatgtggtttacaataataatgtgcaggtctacaatggctgtggattgaggccttcacaaattgtcaaccaacctagggttgacattgcaggagatgattttcgcactttttacactctggttatggtggatccagatgctccaaccccgagcaacccaaatctgagggagtatctccattggctggtcacagatatccctgcaaccacagaagcaacctttggcaatgaaattgtaagttatgagagaccacaaccttcattgggaattcatcgctatattttcgtgttgtttcggcaattggatcgagaggttgtgaatgctcctgatataattgattctcgtgagaattttaacactagagactttgcaaggtttcacggtctcaatttgcctgttgccgctgtttacttcaattgcaatagggaaggcggtaccggtggccgtcgcctataa

SEQ ID NO: 10 - DNA, 978 bp, Nicotiana sylvestris
  source 1..978: mol_type = other DNA
aatagctacctcctcctatactcatgtaagagaaaaattgagataaatttgtaaaaaattccacaccatcaaaatgtcaagactagatcctttaatagtatctggtgttataggagatgtattggatccatttacaaggtctatagactttaatgttgtttacaataataggatgcaagtctacaatggctgtggtttgaggccttcacaaattgtccaccaacctagggttgacgtgggaggagatgatcttcgcactttttacactctggttatggtggatccagatgctccaaccccgagcaatccaaaccagagggagtatctccactggctggtcacaaatatcccagcaaccacaggagcacacttcgggaatgaaattatacaatacgagagtccacgaccttcattgggaattcatcgctatatttttgtgctgtttcgacaattgactcgagatgttgtgaatgctcctgatataattgattctcgtgagaattttaacacaagagactttgcaaggttttacgatctcaattcgcctgttgctgctatgtacttcaatagcaatagggaaagtggtactggtggccgtcacatataaataagtttaacttatatatatcgctagtgtacggaattttacactatcgagtcacatttatggttcaattaaatctcatattttaaagaaatattttgggtgatttgatagtgtaaaaatatcttatactgacggtatatataatttaaattcaaaaagaaataatgttttaataatatgtcccctcaaaaagaaataatgtctaagtaataagtctcctcaaaattttactggatgtcaacatgaggagtttacgtgttacaaagttacatgtaaaagtattgatgctcatatttatttttaattaaaggtgtatcttattatagaactatacttcagttattcaattttacgtcttaacttttatgttt

SEQ ID NO: 11 - DNA, 674 bp, Cucurbita maxima
  source 1..674: mol_type = other DNA
tatttaaacccactgcacctttgtattccttcaacttcatcttcaaggttgacagacagaggcaaagcaatggggatgggaatgatggaggtccatttgattagtggtaaaggacttcaagcccacgaccctcttaataaaccaattgacccttatgctgagatcaacttcaagggccaagaacgcatgagcaaagttgctaaaaatgcaggaccaaatccattatgggatgagaaatttaaattcctagccgagtaccctgggagcggtggtgacttccatatcttattcaaggtcatggaccatgacgctatagatggtgatgattacattggcgatgtcaaaatagatgtcaagaatttattagcggaaggagttagaaaaggaaagtcggagatgccgcctagaatgtaccatgtcttagcacacaaaatccacttcaagggagaaattgaagttggagtgtccttcaagctgcagggcggcgggggatgtggaggatgtaacccatgggaaaactaagtggcttggcttcttcaaacccattggggaaatgtatgcaaacttgttgctttataccaatttgaatttctacctcttttggtatgttattcagattctaaatgtcaaactcctatgacttaacataaaataaaggctatctttttaaataa

SEQ ID NO: 12 - DNA, 2376 bp, Cucurbita maxima; identical to SEQ ID NO: 2
  source 1..2376: mol_type = other DNA

SEQ ID NO: 13 - DNA, 3012 bp, Cucurbita maxima
  source 1..3012: mol_type = other DNA
ccgaccaatgccttatccctttgcttgccaatcactcccaacacgatgatttccactttataaacctaaaaaatcactgcctccaccaatgatttactcctctccctttcccttcagtataaacttccttcccttctctgtcggttgctttccctcttctgtctctctgtttgcagctcaattttgcttccacttcattattctaatcttttgattctgttgactttctgtttttcatagctgctcgagttcctcctaatagttgaatttttcttgttaactcctgtttctatttggagtagatacctatggatccaaacctcaacggttttccagatcatgaagcagattacaactctgattatctggccctcctaacccaattacatgatctccccaatcgttcgattgatgggtatagattaaacgattggccttctttacccacttcatatccataccctgaattcaataacccatctccagatcatgttcccttcatccagaattccattttcccgagcaagccataccctaatcagttttcttcgccgtcaagtgagaggccgaaaggggagccacattctgggtcggtggggttgaactcagatggaggtgcacaaggggtggctttggggttaagccctggaggtgactcttcctcagatgagagtgacttcaaggaaaccgttctcaagtacataagccaaatgcttatggaagagaatttggaggagatgccctgcatgttttatgatcctatgggacttaaagttacggagaaatcgttctatgacgctcttagtgagaactaccctccttcacctaaccaacctcctctggattgcgagagctctgaggatcaatgggctgttgatcctgtagaacatacgccccctgtacttcgaagactttttcccagtaatgagtatcagtcgaattttgaggttacttctggcaatcaaagcaacctgattactaatagtcatgagttcgtcactgagctgttggctcagaatatctttagtgatagcacatccatcttgcaatatcaaaaagggttggaggaggcaaagaagtttcttccaggaggtgaacagctgaatattgatcttggcagcagcatattgacaggagtggcttctaaggtaatggatacaacagagaaggtgaaaagggagaattcaccgaatggatcgaagggacgaaagaatcgggaacgtgaagatgtagacttagattcagaagaagggaggagaaacaagcatgccaccatttatggggatgaggaagaattatctgagatgtttgataaggttttacttcatgattgtggaggaaatgagacctctgcaaatgggaaattgcagtataatggacaacttcatggatctattgctgggaaaactcgggaaaagaaacaggagaagagaaaagactcggtggatttgagaaatcttctgatattatgcgcacaagctgtgtcttctgatgatcgtaggattgctaatgaactacttaagcagattaggcagcattctacaaccaatggggatggtttccaaagaatggctcatttttttgctaacgctcttgaggctcgcatggttggcactggtacaggaagcagaatccactatgaatcactaattcaaagcaagatttcagcagctgatatgttaaaagcttaccaagcacacttatcatcttgcccttttaagaaactctcgctttttttcatggttaaaatgattttgaaggttgctgataaggctaaaagtcttcatattattgattttggcatttgctatggcttcctgtggccgatgttaattcagtttctttcagaattacctgatggtccacctaaactacgcattactggtattgatcaccctctaccaggatttcgtccagctgaaaagattgatgagtcaggacgtcgtttggcaaaatactgcaagcgctttaacgttccttttcaatatcaagccatagcatcagataactgggaaactatccgaatcgaggatttcaagctcgatagcagtgacgtgctagttgtgaactgtttctacaggtttaacaacctacttgacgaaactgtcgaagaaactagtccaagggatattgttctacgtctagtaagggagatgaatccaaacctctttgtacattctatcactaatggatcctatcatgcgccattcttcataacgcgctttcgagaggcactcttccacttctctgcactgtatgattccttagacgttaatttacctcgtgaaagtgaagagaggatgatgatagagagagagtatctcgggcgccaaatcatgaacgtagtagcatgtgagggtgctcagagggttgagaggcctgaaacctataagcaatggcaggttcggtgtattagggcaggtttcaggcagcttcctttggacaaggagatgatgaacaagtttaggagcaagttaacaacccattaccacaaagattttgtactggatgaagataatggctggatacttcaagggtggaaaggccgtattgtctatgcttcttgttgttgggtgccagcatcataggtatactccttgctgtaacttgggacacaagaatgagactccagagtcaagacgagaggcgcattcaatacgttccctctcagatgacagagattgaagcagaactagtgtgagatatttgtacttctctctgctatgggaagtttgaaggtatctgttcgaaggccaagttctgaacaagaggctgcttgtatcagtctctctcttgttagttctatggaaagcaataccttgtcattctgagagggactagaataatgatgtcatgaactgtgggaactaaacatgtatgatcaggagaactgttgtatcttgtctatgaaaatggtagatctgtaattttgagtctattggcttatcctggaaggctacattactgagtgtactaagattgaattgacatgagaatacagtgcttgggaaa

SEQ ID NO: 14 - DNA, 1493 bp, Cucurbita maxima
  source 1..1493: mol_type = other DNA
ttgaagtgcacagagaggtgtgtctcctccaatagctcacaaaatgcctttattcccatcaatctcaccacttctggttatccattttgatcgccccttcatccctccactcactcctttcccttctcctcctattcctctctctaaatcccagtcagaaacaataaaacaaaattaactcacacccatggtgcattgaacgaggttccattccacttctccatctccttggaaacccaacaataaagggttcatagggtttgttcctatcactgtttttgtgtagcgattgacatctgcccgtgtgacaataaccccaagtacgaccaaaaacaccctcttttactgtgtctcttaagcttaaatagcgggtgaaaaggttagccagaccatcatggaccgaggcttgatcatgttcgatcataacaacaacaacgacagcgatagcaacaacaagaagatgtttctgccttacaatgatcatgttcaaacttgtagagctaaacttatggctcatccgttgtttcctcgtctattggcttcctatgtgaattgtcagaaggtgggtgcaccgccggatgtggtggcgaggttagaacaggcttgtacggtggctgctgggaattgtaggacggtggcacgtggggatgatccggggttggatcagttcatggaggcttattgtgagatgttgagtaaatatgaacaagagcttactaagccttttaaagaagctatggttttcttttcgaggattgagtctcagcttaaagccctagctgcttcctcttcttcttcggatggttatgagttggtcgggcaaaacgagtgttcgaaggagatcgaggtcgttgatatgaacgaaaactacatcgacccacaagccgaagagaaggaactcaagggccaacttctacgcaagtacagcggatacctcggtagcctaaagcaagagtttcttaagaagaagaagaatgggaagttgcctaaagaagctcggcagcagctgcttgactggtggagtagacactacaaatggccatatccatcggagtcgcaaaaggtggcgttggccgagtcgacggggctggacttgaagcagatcaataattggttcattaaccaaaggaagcgccattggaagccatcggaggatatgcagtttgtggtgatggatgctgctcatccacactactatttggataatcttatatgcaatccattctctatggactgttcttccactctcttctgattacatatcattttaatgttggtggtttgtttatgttctaagtcatttccattttgttttgaattgttctacatgttgaactcaaaccctttctttgtttcttccttcttgattcttggtctagggttcttgtattgtatgcgtttgttttagtactaatttagctcggttcacattatttaacttttcgaagtttttttttttttaaagtacaatggaaaatctcgttccttgatta

SEQ ID NO: 15 - DNA, 1844 bp, Cucurbita maxima
  source 1..1844: mol_type = other DNA
tcacacttccattcagaagaaaaaaagccgagagagagagagagggagagagcgtggagagggataggctctgtgtctgtgtcgtctgttgtgttggagggctacgaatccacaacagaatcacacacccccttcaaaccaaattcaatcacagactcccctccgcctccaaccaaattctctcagacgacttcacttgtgtgttcattgttttcctctctttcaatcacaacctcgtcgataagatacttcaccaggggggttttgtggagagatttatgtcttctgagtttcatctgtcctctggagctttgaagcagctctgcctttgttttttgtctccgttcttcacccttcacatgcaatttcgttctcgtcaacaacaaccttttcaacccttttttctgcaattaacatcaaatcggtcttgttctggagtgaagaggtttcaagcttctacaagataccataatctcttctcaattttggttcatttttgtttcagggttttgggttttcgttgacattgaagatcgctaaaacttcctctgttctcctgccttcgtttattcattcattatattctgaggttccgaaggcagtttccaaatcaagatatgtatggtaaggaggagaagaaaatgaagcaactaagagaaggaaagtccatttctatgaggaaagttcgtgtactgtttcacgatcctgatgccaccgactactcaagtgaagaagatgaacatgtcaatcaagatgctaaaaacattgtctgggagatatcagtgccagatatccgccataagatatccaaggagagctcacatggaggcaaaattagaaccaaggctgaagtcaaaccaagcagtagcagaacacaaagatcgtcttcgatgtataaaggcgttcgacgaaggaaatgggggaaatatgcagcagagattcgagatccgtttcgtggccgaagattgtggcttggtacttataacaccgctgaggaagctgctgttgcttatcagagaaagaagcacgagttcgaaagtatacaatcgatggcgaagggcagtggtgagctcggccgagagcaacgggatggatcagttgctgattacacagatgaatctgaaaacattatagctttgttttctcatccttcaccatcatctgtacttgatgtttgttgtgaaagtttatgcagcaatgtggatcaaactatggaacattcaatcactgaaggatgtatgcctgtggtagatgatgaagacatcctgaaatcgatctatgaagatgagcagtccattttagacatcgtggaagcgaccccgaccccgaccccgacctcgataacactaccgcggttagatactcaggaattggactttcaagttcttgaagacaattcattgatcttcaatgattttgatcagttctttcatgacacgaacttcagagacaattatactctacacagcatcgaaaacagtgtcggggcgctcgatcttcctccgatggatatggaattcgacaaggagttttcatggtttgacgatacgatgagtatatcatgcatgtaagttttgcagttaggagatagtaatagtcttaagagcagtaaaaaaagaaaaaaaacaaccattgtgaggatgcaatctgtggtaatgtttgtagagaaagagagagagagttcttgggtttttgtgtgtgcttttagagccgtccaagacatttttttggcaactttcgtttgtactgctaagtttacataactattatgaatgcaagttctggtaaggaagcctttggcggctgctgctgctgctctgcaccaatgcaaattcattcagaaa

SEQ ID NO: 16 - DNA, 1305 bp, Cucurbita maxima
  source 1..1305: mol_type = other DNA
tcactctctctttcttctaacttcattctttgcttcttctctctctaaaacataaccctctaagggagagaggatatggggcaccattcttgctgcaacaaacagaaggtgaagagagggctgtggtcaccggaggaagatgagaagcttgtcaattacatttctacttatggccatggctgttggagctctgtccctaagcttgctggcctacaacgatgcgggaaaagttgcaggcttcgatggataaattatctaagacccgaccttaaacgtgggagcttctccccccaagaagctgccctcattgttcaacttcatagcattcttggcaacagatgggctcagatagcaaagcaattaccgggaagaacagacaatgaagtgaagaatttttggaattcgaacgtaaaaaagaagctgatctctcatgaggtttcagctttggccacctttcctggccatctccaaaaccctaatgtttccattaataatgagggcttctttattctcaatgcaaaccctaatttgattctgaccacctcagcccatcaagatcagccatgtcttccaacttcaacgatacaacctggttatgatctcattgattataagcttcagcaattcaccaatctcaatccaaatgccaatttggttcatttgctgccttccttgtccccctcaaccaactttttacctcacagccctccattgtggcagtcattgggcggctaccatccccaatcgctcgactccgatcaactaatggttgcaagcggagagtttgtcactccaacgacgacactacctcaatacgtcgatggttcgattttgcatcccgcaaaattatgcgaagttggtgaggaaaatttttggtcatcaattccacaaatttcagctcctttgcaagatcttgacccatttaccaaattatcaagctttccaaatggttactacccacaagatcaatgcttggcagccaatcaaatggcaatggagcaaattgacaagataatgtcatcgatcttgcagatcccgtcgtgctcctcgtcgtcgtcattgttgggtctatcggaattatcgagcaatctagtttcgttaagctggggagtgtaggatacgtaggtacatcatagtatatgtgtaggtaagtaataattgaaaaaaaaatgcaattattttggtttgatatgtatgtttgagaaggtaagaggtagtccatagggaaagaacctaaaagttgctagttcaggttgtttacaaagtaatgcttgatcaagtactataaatacattgccttgtttc

SEQ ID NO: 17 - protein, 14 aa, synthetic construct
  REGION 1..14: note = NLS
MEDPTMAPKKKRKV

SEQ ID NO: 18 - protein, 7 aa, synthetic construct
  REGION 1..7: note = NLS
PKKKRKV

SEQ ID NO: 19 - protein, 16 aa, synthetic construct
  REGION 1..16: note = NLS
KRPAATKKAGQAKKKK

SEQ ID NO: 20 - protein, 1368 aa, synthetic construct [sequence omitted]

SEQ ID NO: 21 - DNA, 4104 bp, synthetic construct
  misc_feature 1..4104: note = Cas9
  source 1..4104: mol_type = other DNA
atggataaaaaatatagcattggtctggatattggtaccaatagcgttggttgggcagttattaccgatgaatataaagttccgagcaaaaaatttaaagttctgggtaataccgatcgtcatagcattaaaaaaaatctgattggtgcactgctgtttgatagcggtgaaaccgcagaagcaacccgtctgaaacgtaccgcacgtcgtcgttatacccgtcgtaaaaatcgtatttgttatctgcaggaaatttttagcaatgaaatggcaaaagttgatgatagcttttttcatcgtctggaagaaagctttctggttgaagaagataaaaaacatgaacgtcatccgatttttggtaatattgttgatgaagttgcatatcatgaaaaatatccgaccatttatcatctgcgtaaaaaactggttgatagcaccgataaagcagatctgcgtctgatttatctggcactggcacatatgattaaatttcgtggtcattttctgattgaaggtgatctgaatccggataatagcgatgttgataaactgtttattcagctggttcagacctataatcagctgtttgaagaaaatccgattaatgcaagcggtgttgatgcaaaagcaattctgagcgcacgtctgagcaaaagccgtcgtctggaaaatctgattgcacagctgccgggtgaaaaaaaaaatggtctgtttggtaatctgattgcactgagcctgggtctgaccccgaattttaaaagcaattttgatctggcagaagatgcaaaactgcagctgagcaaagatacctatgatgatgatctggataatctgctggcacagattggtgatcagtatgcagatctgtttctggcagcaaaaaatctgagcgatgcaattctgctgagcgatattctgcgtgttaataccgaaattaccaaagcaccgctgagcgcaagcatgattaaacgttatgatgaacatcatcaggatctgaccctgctgaaagcactggttcgtcagcagctgccggaaaaatataaagaaattttttttgatcagagcaaaaatggttatgcaggttatattgatggtggtgcaagccaggaagaattttataaatttattaaaccgattctggaaaaaatggatggtaccgaagaactgctggttaaactgaatcgtgaagatctgctgcgtaaacagcgtacctttgataatggtagcattccgcatcagattcatctgggtgaactgcatgcaattctgcgtcgtcaggaagatttttatccgtttctgaaagataatcgtgaaaaaattgaaaaaattctgacctttcgtattccgtattatgttggtccgctggcacgtggtaatagccgttttgcatggatgacccgtaaaagcgaagaaaccattaccccgtggaattttgaagaagttgttgataaaggtgcaagcgcacagagctttattgaacgtatgaccaattttgataaaaatctgccgaatgaaaaagttctgccgaaacatagcctgctgtatgaatattttaccgtttataatgaactgaccaaagttaaatatgttaccgaaggtatgcgtaaaccggcatttctgagcggtgaacagaaaaaagcaattgttgatctgctgtttaaaaccaatcgtaaagttaccgttaaacagctgaaagaagattattttaaaaaaattgaatgttttgatagcgttgaaattagcggtgttgaagatcgttttaatgcaagcctgggtacctatcatgatctgctgaaaattattaaagataaagattttctggataatgaagaaaatgaagatattctggaagatattgttctgaccctgaccctgtttgaagatcgtgaaatgattgaagaacgtctgaaaacctatgcacatctgtttgatgataaagttatgaaacagctgaaacgtcgtcgttataccggttggggtcgtctgagccgtaaactgattaatggtattcgtgataaacagagcggtaaaaccattctggattttctgaaaagcgatggttttgcaaatcgtaattttatgcagctgattcatgatgatagcctgacctttaaagaagatattcagaaagcacaggttagcggtcagggtgatagcctgcatgaacatattgcaaatctggcaggtagcccggcaattaaaaaaggtattctgcagaccgttaaagttgttgatgaactggttaaagttatgggtcgtcataaaccggaaaatattgttattgaaatggcacgtgaaaatcagaccacccagaaaggtcagaaaaatagccgtgaacgtatgaaacgtattgaagaaggtattaaagaactgggtagccagattctgaaagaacatccggttgaaaatacccagctgcagaatgaaaaactgtatctgtattatctgcagaatggtcgtgatatgtatgttgatcaggaactggatattaatcgtctgagcgattatgatgttgatcatattgttccgcagagctttctgaaagatgatagcattgataataaagttctgacccgtagcgataaaaatcgtggtaaaagcgataatgttccgagcgaagaagttgttaaaaaaatgaaaaattattggcgtcagctgctgaatgcaaaactgattacccagcgtaaatttgataatctgaccaaagcagaacgtggtggtctgagcgaactggataaagcaggttttattaaacgtcagctggttgaaacccgtcagattaccaaacatgttgcacagattctggatagccgtatgaataccaaatatgatgaaaatgataaactgattcgtgaagttaaagttattaccctgaaaagcaaactggttagcgattttcgtaaagattttcagttttataaagttcgtgaaattaataattatcatcatgcacatgatgcatatctgaatgcagttgttggtaccgcactgattaaaaaatatccgaaactggaaagcgaatttgtttatggtgattataaagtttatgatgttcgtaaaatgattgcaaaaagcgaacaggaaattggtaaagcaaccgcaaaatattttttttatagcaatattatgaatttttttaaaaccgaaattaccctggcaaatggtgaaattcgtaaacgtccgctgattgaaaccaatggtgaaaccggtgaaattgtttgggataaaggtcgtgattttgcaaccgttcgtaaagttctgagcatgccgcaggttaatattgttaaaaaaaccgaagttcagaccggtggttttagcaaagaaagcattctgccgaaacgtaatagcgataaactgattgcacgtaaaaaagattgggatccgaaaaaatatggtggttttgatagcccgaccgttgcatatagcgttctggttgttgcaaaagttgaaaaaggtaaaagcaaaaaactgaaaagcgttaaagaactgctgggtattaccattatggaacgtagcagctttgaaaaaaatccgattgattttctggaagcaaaaggttataaagaagttaaaaaagatctgattattaaactgccgaaatatagcctgtttgaactggaaaatggtcgtaaacgtatgctggcaagcgcaggtgaactgcagaaaggtaatgaactggcactgccgagcaaatatgttaattttctgtatctggcaagccattatgaaaaactgaaaggtagcccggaagataatgaacagaaacagctgtttgttgaacagcataaacattatctggatgaaattattgaacagattagcgaatttagcaaacgtgttattctggcagatgcaaatctggataaagttctgagcgcatataataaacatcgtgataaaccgattcgtgaacaggcagaaaatattattcatctgtttaccctgaccaatctgggtgcaccggcagcatttaaatattttgataccaccattgatcgtaaacgttataccagcaccaaagaagttctggatgcaaccctgattcatcagagcattaccggtctgtatgaaacccgtattgatctgagccagctgggtggtgat

SEQ ID NO: 22 - protein, 1082 aa, unidentified
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 23 - protein, 1300 aa, synthetic construct
  REGION 1..1300: note = Cpf1
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 24 - DNA, 3900 bp, synthetic construct
  misc_feature 1..3900: note = Cpf1
  source 1..3900: mol_type = other DNA
atgagcatttatcaggaatttgttaataaatatagcctgagcaaaaccctgcgttttgaactgattccgcagggtaaaaccctggaaaatattaaagcacgtggtctgattctggatgatgaaaaacgtgcaaaagattataaaaaagcaaaacagattattgataaatatcatcagttttttattgaagaaattctgagcagcgtttgtattagcgaagatctgctgcagaattatagcgatgtttattttaaactgaaaaaaagcgatgatgataatctgcagaaagattttaaaagcgcaaaagataccattaaaaaacagattagcgaatatattaaagatagcgaaaaatttaaaaatctgtttaatcagaatctgattgatgcaaaaaaaggtcaggaaagcgatctgattctgtggctgaaacagagcaaagataatggtattgaactgtttaaagcaaatagcgatattaccgatattgatgaagcactggaaattattaaaagctttaaaggttggaccacctattttaaaggttttcatgaaaatcgtaaaaatgtttatagcagcaatgatattccgaccagcattatttatcgtattgttgatgataatctgccgaaatttctggaaaataaagcaaaatatgaaagcctgaaagataaagcaccggaagcaattaattatgaacagattaaaaaagatctggcagaagaactgacctttgatattgattataaaaccagcgaagttaatcagcgtgtttttagcctggatgaagtttttgaaattgcaaattttaataattatctgaatcagagcggtattaccaaatttaataccattattggtggtaaatttgttaatggtgaaaataccaaacgtaaaggtattaatgaatatattaatctgtatagccagcagattaatgataaaaccctgaaaaaatataaaatgagcgttctgtttaaacagattctgagcgataccgaaagcaaaagctttgttattgataaactggaagatgatagcgatgttgttaccaccatgcagagcttttatgaacagattgcagcatttaaaaccgttgaagaaaaaagcattaaagaaaccctgagcctgctgtttgatgatctgaaagcacagaaactggatctgagcaaaatttattttaaaaatgataaaagcctgaccgatctgagccagcaggtttttgatgattatagcgttattggtaccgcagttctggaatatattacccagcagattgcaccgaaaaatctggataatccgagcaaaaaagaacaggaactgattgcaaaaaaaaccgaaaaagcaaaatatctgagcctggaaaccattaaactggcactggaagaatttaataaacatcgtgatattgataaacagtgtcgttttgaagaaattctggcaaattttgcagcaattccgatgatttttgatgaaattgcacagaataaagataatctggcacagattagcattaaatatcagaatcagggtaaaaaagatctgctgcaggcaagcgcagaagatgatgttaaagcaattaaagatctgctggatcagaccaataatctgctgcataaactgaaaatttttcatattagccagagcgaagataaagcaaatattctggataaagatgaacatttttatctggtttttgaagaatgttattttgaactggcaaatattgttccgctgtataataaaattcgtaattatattacccagaaaccgtatagcgatgaaaaatttaaactgaattttgaaaatagcaccctggcaaatggttgggataaaaataaagaaccggataataccgcaattctgtttattaaagatgataaatattatctgggtgttatgaataaaaaaaataataaaatttttgatgataaagcaattaaagaaaataaaggtgaaggttataaaaaaattgtttataaactgctgccgggtgcaaataaaatgctgccgaaagttttttttagcgcaaaaagcattaaattttataatccgagcgaagatattctgcgtattcgtaatcatagcacccataccaaaaatggtagcccgcagaaaggttatgaaaaatttgaatttaatattgaagattgtcgtaaatttattgatttttataaacagagcattagcaaacatccggaatggaaagattttggttttcgttttagcgatacccagcgttataatagcattgatgaattttatcgtgaagttgaaaatcagggttataaactgacctttgaaaatattagcgaaagctatattgatagcgttgttaatcagggtaaactgtatctgtttcagatttataataaagattttagcgcatatagcaaaggtcgtccgaatctgcataccctgtattggaaagcactgtttgatgaacgtaatctgcaggatgttgtttataaactgaatggtgaagcagaactgttttatcgtaaacagagcattccgaaaaaaattacccatccggcaaaagaagcaattgcaaataaaaataaagataatccgaaaaaagaaagcgtttttgaatatgatctgattaaagataaacgttttaccgaagataaatttttttttcattgtccgattaccattaattttaaaagcagcggtgcaaataaatttaatgatgaaattaatctgctgctgaaagaaaaagcaaatgatgttcatattctgagcattgatcgtggtgaacgtcatctggcatattataccctggttgatggtaaaggtaatattattaaacaggatacctttaatattattggtaatgatcgtatgaaaaccaattatcatgataaactggcagcaattgaaaaagatcgtgatagcgcacgtaaagattggaaaaaaattaataatattaaagaaatgaaagaaggttatctgagccaggttgttcatgaaattgcaaaactggttattgaatataatgcaattgttgtttttgaagatctgaattttggttttaaacgtggtcgttttaaagttgaaaaacaggtttatcagaaactggaaaaaatgctgattgaaaaactgaattatctggtttttaaagataatgaatttgataaaaccggtggtgttctgcgtgcatatcagctgaccgcaccgtttgaaacctttaaaaaaatgggtaaacagaccggtattatttattatgttccggcaggttttaccagcaaaatttgtccggttaccggttttgttaatcagctgtatccgaaatatgaaagcgttagcaaaagccaggaattttttagcaaatttgataaaatttgttataatctggataaaggttattttgaatttagctttgattataaaaattttggtgataaagcagcaaaaggtaaatggaccattgcaagctttggtagccgtctgattaattttcgtaatagcgataaaaatcataattgggatacccgtgaagtttatccgaccaaagaactggaaaaactgctgaaagattatagcattgaatatggtcatggtgaatgtattaaagcagcaatttgtggtgaaagcgataaaaaattttttgcaaaactgaccagcgttctgaataccattctgcagatgcgtaatagcaaaaccggtaccgaactggattatctgattagcccggttgcagatgttaatggtaatttttttgatagccgtcaggcaccgaaaaatatgccgcaggatgcagatgcaaatggtgcatatcatattggtctgaaaggtctgatgctgctgggtcgtattaaaaataatcaggaaggtaaaaaactgaatctggttattaaaaatgaagaatattttgaatttgttcagaatcgtaataat

SEQ ID NO: 25 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 26 - protein, 1274 aa, synthetic construct
  REGION 1..1274: note = MAD7-NLS
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLSGGSPKKKRKV

SEQ ID NO: 27 - RNA, 23 nt, synthetic construct
  misc_feature 1..23: note = cleavable spacer
  source 1..23: mol_type = other RNA
tcgcggccgggtacgtgttgagc

SEQ ID NO: 28 - DNA, 2067 bp, Solanum tuberosum
  source 1..2067: mol_type = other DNA
atgtactatcaaggaacctcggataatactaatatacaagctgatcatcaacaacgtcataatcatgggaatagtaataataataatattcagacactttatttgatgaaccctaacaattatatgcaaggctacactacttctgacacacagcagcagcagcagttacttttcctgaattcttcaccagcagcaagcaacgcgctttgccatgcgaatatacaacacgcgccgctgcaacagcagcactttgtcggtgtgcctcttccggcagtaagtttgcacgatcagatcaatcatcatggacttttacagcgcatgtggaacaaccaagatcaatctcagcaggtgatagtaccatcgtcgacgggggtttctgccacgtcatgtggcgggatcaccacggacttggcgtctcaattggcgtttcagaggccgattccgacaccacaacaccgacagcagcaacaacagcaaggcggtctatctctaagcctttctcctcagctacaacagcaaattagtttcaataacaatatttcatcctcatcaccaaggacaaataatgttactattaggggaacattagatggaagttctagcaacatggttttaggctctaagtatctgaaagctgcacaagagcttcttgatgaagttgttaatattgttggaaaaagcatcaaaggagatgatcaaaagaaggataattcaatgaataaagaatcaatgcctttggctagtgatgtcaacactaatagttctggtggtggtgaaagtagcagcaggcagaaaaatgaagttgctgttgagcttacaactgctcaaagacaagaacttcaaatgaaaaaagccaagcttcttgccatgcttgaagaggtggagcaaaggtacagacagtaccatcaccaaatgcaaataattgtattatcatttgagcaagtagcaggaattggatcagccaaatcatacactcaattagctttgcatgcaatttcgaagcaattcagatgcctaaaggatgcaattgctgagcaagtaaaggcgacgagcaagagtttaggtgaagaggaaggcttgggagggaaaatcgaaggctcaagactcaaatttgtggaccatcatctaaggcaacaacgcgcgctgcaacagataggaatgatgcaaccaaatgcttggagaccccaaagaggtttacctgaaagagctgtctctgtccttcgtgcttggcttttcgagcattttcttcatccttacccaaaggattcagacaaaatcatgcttgctaagcaaacggggctaacaaggagccaggtgtctaactggttcataaatgctcgagttcgattatggaagccaatggtagaagaaatgtacttggaagaagtgaagaatcaagaacaaaacagtactaatacttcaggagataacaaaaacaaagagaccaatataagtgctccaaatgaagagaaacatccaattattactagcagcttattacaagatggtattactactactcaagcagaaatttctacctcaactatttcaacttcccctactgcaggtgcttcacttcatcatgctcacaatttctccttccttggttcattcaacatggataatactactactactgttgatcatattgaaaacaacgcgaaaaagcaaagaaatgacatgcacaagttttctccaagtagtattctttcatctgttgacatggaagccaaagctagagaatcatcaaataaagggtttactaatcctttaatggcagcatacgcgatgggagattttggaaggtttgatcctcatgatcaacaaatgaccgcgaattttcatggaaataatggtgtctctcttactttaggacttcctccttctgaaaacctagccatgccagtgagccaacaaaattacctttctaatgacttgggaagtaggtctgaaatggggagtcattacaatagaatgggatatgaaaacattgattttcagagtgggaataagcgatttccgactcaactattaccagattttgttacaggtaatctaggaacatga

SEQ ID NO: 29 - DNA, 20 bp, synthetic construct
  misc_feature 1..20: note = GFP forward primer
  source 1..20: mol_type = other DNA
ggtcttgtagttgccgtcgt

SEQ ID NO: 30 - DNA, 20 bp, synthetic construct
  misc_feature 1..20: note = GFP reverse primer
  source 1..20: mol_type = other DNA
tcgtgaccaccctgacctac

SEQ ID NO: 31 - DNA, 20 bp, synthetic construct
  misc_feature 1..20: note = Cas9 forward primer
  source 1..20: mol_type = other DNA
aagaggcagcttgtggaaac

SEQ ID NO: 32 - DNA, 20 bp, synthetic construct
  misc_feature 1..20: note = Cas9 reverse primer
  source 1..20: mol_type = other DNA
acacgagctttgacttgagg

SEQ ID NO: 33 - DNA, 21 bp, synthetic construct
  misc_feature 1..21: note = tubulin forward primer
  source 1..21: mol_type = other DNA
caagtgttgctgaggtcttct

SEQ ID NO: 34 - DNA, 22 bp, synthetic construct
  misc_feature 1..22: note = tubulin reverse primer
  source 1..22: mol_type = other DNA
agaactctccttcctccatacc

SEQ ID NO: 35 - DNA, 20 bp, synthetic construct
  misc_feature 1..20: note = Cas9 forward primer
  source 1..20: mol_type = other DNA
agttgttcatccagctcgtg

SEQ ID NO: 36 - DNA, 20 bp, synthetic construct
  misc_feature 1..20: note = Cas9 reverse primer
  source 1..20: mol_type = other DNA
ggtcttgtagttgccgtcgt

SEQ ID NO: 37 - DNA, 22 bp, synthetic construct
  misc_feature 1..22: note = forward primer
  source 1..22: mol_type = other DNA
gctaagaacgctggacctaatg

SEQ ID NO: 38 - DNA, 20 bp, synthetic construct
  misc_feature 1..20: note = reverse primer
  source 1..20: mol_type = other DNA
tgggtgtgcctttctgaatg